SEQUENCE LISTING (20 sequences)

SEQ ID NO: 1 - protein, 1008 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 2 - protein, 212 aa, Bacillus subtilis [sequence omitted]

SEQ ID NO: 3 - DNA, 45 bp, Artificial Sequence
  Synthetic primer
ttatggttcacggtattggagttttagagctagaaatagcaagtt

SEQ ID NO: 4 - DNA, 44 bp, Artificial Sequence
  Synthetic primer
gatagcagaagaaaatggaggaattgtcagaccaagtttactca

SEQ ID NO: 5 - DNA, 44 bp, Artificial Sequence
  Synthetic primer
tgagtaaacttggtctgacaattcctccattttcttctgctatc

SEQ ID NO: 6 - DNA, 39 bp, Artificial Sequence
  Synthetic primer
tccaataccgtgaaccataatccacacattatgccacac

SEQ ID NO: 7 - DNA, 20 bp, Artificial Sequence
  Synthetic primer
ttatggttcacggtattgga

SEQ ID NO: 8 - DNA, 64 bp, Artificial Sequence
  Synthetic primer
cttttgcaggacgtgcatttcagacttgtgtaaagcctggggtgcctaatgagtgaggtaactc

SEQ ID NO: 9 - DNA, 40 bp, Artificial Sequence
  Synthetic primer
gtagtaagaactattcatagagtgaatcgaaaacaatacg

SEQ ID NO: 10 - DNA, 40 bp, Artificial Sequence
  Synthetic primer
cgtattgttttcgattcactctatgaatagttcttactac

SEQ ID NO: 11 - DNA, 61 bp, Artificial Sequence
  Synthetic primer
ctatggttcttctcaaatgtcacgactttcgctttcttcccttcctttctcgccacgttcg

SEQ ID NO: 12 - DNA, 52 bp, Artificial Sequence
  Synthetic primer
cgagaaaggaagggaagaaagcgaaagtcgtgacatttgagaagaaccatag

SEQ ID NO: 13 - DNA, 54 bp, Artificial Sequence
  Synthetic primer
cttcaaggttttgtttttcattaattctaagattcagaggtcattattggtcct

SEQ ID NO: 14 - DNA, 57 bp, Artificial Sequence
  Synthetic primer
aggaccaataatgacctctgaatcttagaattaatgaaaaacaaaaccttgaagaat

SEQ ID NO: 15 - DNA, 52 bp, Artificial Sequence
  Synthetic primer
tcattaggcaccccaggctttacacaagtctgaaatgcacgtcctgcaaaag

SEQ ID NO: 16 - DNA, 34 bp, Artificial Sequence
  Synthetic primer
cgtgacatttgagaagaaccatagtacaacggtg

SEQ ID NO: 17 - DNA, 28 bp, Artificial Sequence
  Synthetic primer
gtctgaaatgcacgtcctgcaaaagaag

SEQ ID NO: 18 - DNA, 23 bp, Artificial Sequence
  Synthetic primer
gtcggttcgatgagaccttccac

SEQ ID NO: 19 - DNA, 23 bp, Artificial Sequence
  Synthetic primer
ctgccatgattctacgatctcac

SEQ ID NO: 20 - DNA, 639 bp, Bacillus subtilis
atgaaatttgtaaaaagaaggatcattgcacttgtaacaattttgatgctgtctgttacatcgctgtttgcgttgcagccgtcagcaaaagccgctgaacacaatccagtcgttatggttcacggtattggaggggcatcattcaattttgcgggaattaagagctatctcgtatctcagggctggtcgcgggacaagctgtatgcagttgatttttgggacaagacaggcacaaattataacaatggaccggtattatcacgatttgtgcaaaaggttttagatgaaacgggtgcgaaaaaagtggatattgtcgctcacagcatggggggcgcgaacacactttactacataaaaaatctggacggcggaaataaagttgcaaacgtcgtgacgcttggcggcgcgaaccgtttgacgacaggcaaggcgcttccgggaacagatccaaatcaaaagattttatacacatccatttacagcagtgccgatatgattgtcatgaattacttatcaagattagatggtgctagaaacgttcaaatccatggcgttggacacatcggccttctgtacagcagccaagtcaacagcctgattaaagaagggctgaacggcgggggccagaatacgaattaa

(19) United States
(12) Patent Application Publication
(10) Pub. No.: US 2022/0132878 A1
(43) Pub. Date: May 5, 2022

(54) METHOD FOR REDUCING LACTOSE AT HIGH TEMPERATURES

(71) Applicant: DUPONT NUTRITION BIOSCIENCES APS, COPENHAGEN K (DK)

(72) Inventors: Karina Hansen KJAER, Brabrand (DK); Jacob Flyvholm CRAMER, Palo Alto, CA (US); Tina Krogh JENSEN, Skanderborg (DK); Tomoharu SATO, PALO ALTO, CA (US); Cristina BONGIORNI, PALO ALTO, CA (US)

(21) Appl. No.: 17/431,557

(22) PCT Filed: Feb. 27, 2020
(86) PCT No.: PCT/US2020/020108

§ 371 Date: Aug. 17, 2021

Related U.S. Application Data
(60) Provisional application No. 62/811,722, filed on Feb. 28, 2019.

(51) Int. Cl. A23C 9/12; C12N 9/24; C12P 19/04; C12P 19/14; A23C 9/127

(52) U.S. Cl. CPC A23C 9/1206; C12N 9/2402; C12P 19/04; A23G 9/40; A23C 9/1275; C12Y 302/01108; C12P 19/14

(57) ABSTRACT

This specification relates to a method for reducing the amount of lactose in a milk-based substrate containing lactose, wherein said method comprises contacting said substrate with an enzyme having neutral lactase activity at a temperature of more than about 50° C., and wherein said lactase reduces the amount of lactose in said substrate by at least about 70%.

### FIELD OF THE INVENTION

The application relates to methods for reducing the amount of lactose in milk-based substrates containing lactose by contacting the substrate with an enzyme having neutral lactase activity, more particularly at higher temperatures. This specification also refers to milk-based substrates and dairy products, such as lactose reduced or lactose free, obtained or obtainable by the methods disclosed in this specification. The specification also refers to an enzyme preparation comprising an enzyme having neutral lactase activity as disclosed in this specification, nucleic acid molecules encoding the enzyme, vectors comprising the nucleic acids molecules, cells capable of expressing the enzyme, and the use thereof for preparing milk-based substrates and dairy products. The specification also relates to a concentrated and purified lactase efficient in lactose hydrolysis at 50-65° C. which can be used for the preparation of lactose reduced or lactose free dairy products.

### BACKGROUND OF THE INVENTION

Lactose intolerant people have difficulties in digesting dairy products with high lactose levels. It is estimated that about 70% of the world's population has a limited ability to digest lactose. Accordingly, there is a growing demand for dairy food products that contain no or only low levels of lactose. The commercial use of lactases is to break down lactose in dairy products. A typical process for production of pasteurized milk with reduced lactose comprises addition of the lactase enzyme to the milk followed by prolonged incubation (10-48 hours, often 24 hours) at temperatures around 6° C. Lactases have been isolated from a large variety of organisms, including microorganisms like Kluyveromyces. Bacillus and Bifidobacterium. Kluyveromyces, especially K. fragilis and K. lactis, and other fungi such as those of the genera Candida, Torula and Torulopsis, are a common source of fungal lactases, whereas B. coagulans and B. circulans are well known sources for bacterial lactases. Several commercial lactase preparations derived from these organisms are available such as Lactozym® (available from Novozymes, Denmark), HA-Lactase (available from Chr. Hansen, Denmark) and Maxilact® (available from DSM, the Netherlands), these are all from K. lactis. Also, a few Bifidobacterium bifidum lactases are commercially available such as Saphera (available from Novozymes, Denmark) and NOLA Fit (available from Chr. Hansen, Denmark). All these lactases are so-called neutral lactases having a pH optimum between pH 6 and pH 8, as well as a temperature optimum around 37° C. These lactases are not suitable for hydrolysis of lactose in milk substrates performed at high temperatures, which would in some cases be beneficial in order to keep the microbial count low and thus ensure high milk quality.

Only few commercial lactase preparations are available with a temperature optimum above 50° C., but they are GOS producing lactases and/or acid lactases such as the Lactoles, a commercial Bacillus lactase from Daiwa Kasei (Japan), the FoodPro GOS, a Bifidumbacterium bifidum lactase from DuPont and the NutribioGOS an acid lactase from Aspergillus oryzae, which makes them less efficient in production of lactose reduced or lactose free neutral dairy products.

In U.S. Pat. Nos. 4,374,861 and 4,333,954, which relate to ice cream, lactose is hydrolyzed at lower temperatures of 30-50° C. Hydrolysis at these low temperatures risks a high microbial count.

In CZ 201100672, which relates to condensed milk, lactose is hydrolyzed at lower temperatures of 2-50° C. Hydrolysis at these low temperatures is not efficient. In addition, hydrolysis at these low temperatures risks a high microbial count.

The present invention provides a neutral lactase efficient in lactose hydrolysis at 50-65° C. for production of dairy products.

### SUMMARY OF THE INVENTION

Briefly, this specification generally discloses methods for reducing the amount of lactose in milk-based substrates and dairy products containing lactose, as well as milk-based substrates and dairy products produced by the methods.

The specification discloses, in part, enzyme preparations comprising an enzyme having neutral lactase activity as well as vectors and nucleic acid molecules encoding the enzyme and cells capable of expressing the enzyme.

Advantageously, the methods described herein produce milk-based substrates and/or dairy products which are lactose free or have a reduction in the level of lactose. Advantageously the methods result in milk-based substrates and/or dairy products which have lower levels of off-flavor and/or fewer texture defects.

Further benefits of the teachings of this specification will be apparent to one skilled in the art from reading this specification.

### BRIEF DESCRIPTION OF THE DRAWINGS

FIG. 1 depicts nucleotide map of the plasmid pCAS-lipA

FIG. 2 depicts nucleotide map of the plasmid pRS426-lipA

FIG. 3 depicts the relative lactase temperature optimum for the lactase indicated in figure legends, ranging from 5° C. to 70° C. The relative lactase activity for all samples is 100% at 30° C.

FIG. 4 depicts the residual lactase activity in milk-based substrate at high temperature (A: 55° C. and 58° C.) for Experimental Dupont lactase SEQ ID NO: 1 and Nola Fit, as labelled. Residual activity was quantified for up to 360 minutes of incubation.

FIG. 5 (A) depicts the residual lactose in % (w/v) in Arla Mini-milk samples (0.5% fat and 4.8% lactose) added various lactases (see legends) and incubated at 60° C. The residual lactose in the milk samples are shown for 10 to 240 minutes of incubation. (B) depicts the amount of lactose reduction using the lactase of SEQ ID NO:1 at various temperatures.

FIG. 6 depicts recombined sweet condensed milk samples with various addition of lactase after 1 week of storage at room temperature. Skimmed milk powder and water was mixed at 55° C., combined with lactase at 60° C., melted butter oil mixed together with sugar and Recodan (sample 6) at 75° C. following homogenized at 75° C. (only 65° C. for trial 6), 80 bar and then pasteurized at 90° C. 15 seconds. The blank sample 8 show clear lactose crystal formation whereas samples with lactase SEQ ID NO: 1 (2-6) and Recodan (6) showed no lactose crystal formation.

FIG. 7 depicts cooked sweet condensed milk samples stored 6 weeks. Samples with lactase SEQ ID NO: 1 (2-6), and Recodan (6), grinded lactose (7) and blank (8).

FIG. 8 depicts sensory analysis of UHT milk.

### BRIEF DESCRIPTION OF THE SEQUENCE ID NOS

SEQ ID NO:1 is the amino acid sequence of the mature form of B-galactosidase from Lactobacillus delbrueckii bulgaricus, LBul.

SEQ ID NO:2 is the amino acid sequence of Lipase A.

SEQ ID NO:3 is the DNA sequence of CB 1387.

SEQ ID NO:4 is the DNA sequence of CB 1284.

SEQ ID NO:5 is the DNA sequence of CB 1283.

SEQ ID NO:6 is the DNA sequence of CB 1288.

SEQ ID NO:7 is the DNA sequence of the LipA target site primer.

SEQ ID NO:8 is the DNA sequence of DH 18-327F.

SEQ ID NO:9 is the DNA sequence of DH 18-273R.

SEQ ID NO:10 is the DNA sequence of DH 18-272F.

SEQ ID NO:11 is the DNA sequence of DH 18-325R.

SEQ ID NO:12 is the DNA sequence of DH 18-317F.

SEQ ID NO:13 is the DNA sequence of DH 18-320R.

SEQ ID NO:14 is the DNA sequence of DH 18-316F.

SEQ ID NO:15 is the DNA sequence of DH 18-318R.

SEQ ID NO:16 is the DNA sequence of DH 18-343R.

SEQ ID NO:17 is the DNA sequence of DH 18-346R.

SEQ ID NO:18 is the DNA sequence of DH 18-385F.

SEQ ID NO:19 is the DNA sequence of DH 18-386R.

SEQ ID NO:20 is the DNA sequence of lipase A.

### DETAILED DESCRIPTION

This detailed description is intended to acquaint others skilled in the art with Applicant's invention, its principles, and its practical application so that others skilled in the art may adapt and apply Applicant's invention in its numerous forms, as they may be best suited to the requirements of a particular use. This detailed description and its specific examples, while indicating certain aspects, are intended for purposes of illustration only. This specification, therefore, is not limited to the described aspects, and may be variously modified.

This specification discloses a method for reducing the amount of lactose in a milk-based substrate containing lactose, wherein said method comprises contacting said substrate with an enzyme having neutral lactase activity at a temperature of more than about 50° C., and wherein said lactase reduces the amount of lactose in said substrate by at least about 70%.

This specification also discloses a milk-based substrate with reduced lactose content obtained or obtainable by the method described above (or elsewhere in the specification).

This specification further discloses a method for producing a dairy product wherein said method comprises contacting said dairy product with an enzyme having neutral lactase activity at a temperature of more than about 50° C., and wherein said lactase reduces the amount of lactose in said dairy product by at least about 70%.

This specification also discloses a dairy product obtained or obtainable by the method described above (or elsewhere in the specification).

This specification further discloses, in part, an enzyme preparation comprising an enzyme having neutral lactase activity, wherein said enzyme can reduce the amount of lactose in a substrate by at least 70% at a temperature of 50° C. or more.

This specification also discloses, in part, an enzyme preparation comprising an enzyme having neutral lactase activity, wherein said enzyme can reduce the amount of lactose in a substrate by at least 70% at a temperature of 50° C. or more, wherein said enzyme is not that of SEQ ID NO:1.

This specification further discloses, in part, a nucleic acid molecule encoding an enzyme having neutral lactase activity or lactase active fragment thereof as described herein.

This specification also discloses, in part, an expression vector comprising a nucleic acid molecule as described above (or elsewhere in the specification), or capable of expressing an having neutral lactase activity or lactase active fragment thereof as described herein.

This specification also discloses, in part, a cell capable of expressing an enzyme having neutral lactase activity or lactase active fragment thereof as described herein.

This specification further discloses, in part, a method of expressing an enzyme, comprising providing a cell as described above (or elsewhere in the specification) and expressing the enzyme from the cell, and optionally purifying the enzyme.

This specification also discloses, in part, an enzyme having neutral lactase activity as described hereon which has a half-life in milk of more than 4 hours at 55° C., or more than 1 hour at 58° C.

This specification further discloses, in part, use of an enzyme preparation as described herein for preparing a dairy product.

This specification further discloses, in part, a bacterial expression host capable of expressing an enzyme having neutral lactase activity as described herein wherein the host cell comprises a genetic modification which reduces or eliminates lipase activity, preferably lipase A activity.

This specification further discloses, in part, an enzyme having neutral lactase activity produced by a bacterial expression host as described herein.

This specification further discloses, in part, an enzyme having neutral lactase activity produced by a bacterial expression host as described herein wherein said enzyme is an enzyme preparation as defined herein.

This specification also discloses, in part, an enzyme a dairy product comprising the bacterial expression host as described herein.

This specification further discloses, in part, the use of the bacterial expression host as described herein for preparing a dairy product.

This specification further discloses, in part, a method for producing a lactose reduced or lactose free milk shake, ice cream, reconstituted milk product, desserts, pudding, condensed milk, sweetened condensed milk, Ryazhenka, Dulce de Leche or milk based powder, said method comprising contacting a milk-based substrate with a lactase at 50-65° C.

This specification also discloses, in part, a method for producing milk-based powder or whey powder with a reduced lactose content in which an enzyme having neutral lactase activity is added for hydrolyzation during the evaporation/condensing process.

This specification further discloses, in part, a method for production of a lactose free dairy product from a milk-based substrate with an enzyme having neutral lactase activity, wherein more than 20% activity remains in the milk-based substrate after pasteurization at 72° C. for 15 seconds.

This specification further discloses, in part, a method for producing a fermented dairy product comprising adding a lactase after pasteurization and homogenization, for example during cooling to the fermentation temperature.

The specification further discloses, in part, a method for producing a fermented dairy product comprising adding a lactase after pasteurization and homogenization, for example while cooling only to about 50-65° C. Further cooling may then occur, and starter cultures added when the temperature is around 45° C.

The specification further discloses, in part, a method for in situ GOS production in a milk-based substrate comprising at least 4.7% (w/w) lactose, comprising contacting said substrate with a lactase having neutral lactase activity wherein more than 30% of said lactose is converted into TGOS.

The specification further discloses, in part, a method for in situ GOS production wherein the milk-based substrate comprises between 6-40% lactose (w/w) and wherein more than 40% of the lactose is converted into TGOS.

The specification further discloses, in part, a method for reducing the amount of sugar in a milk-based substrate comprising contacting said substrate with an enzyme having neutral lactase activity wherein lactose is converted into GOS fibers (DP3+).

The specification further discloses, in part, a method for the production of a lactose free dairy product from a milk-based substrate having the steps of providing a milk-based substrate; adding an enzyme having neutral lactase activity to the milk based substrate; pasteurizing the milk-based substrate wherein the enzyme retains a substantial amount of activity after said pasteurization step and storing the resulting milk-based substrate for a sufficient time to produce a lactose free dairy product.

### Enzyme Having Neutral Lactase Activity

The terms “enzyme having neutral lactase activity” and “lactase” may be used interchangeably herein.

An enzyme having neutral lactase activity is any enzyme which is capable of hydrolysing the disaccharide lactose into constituent galactose and glucose monomers. In addition, an enzyme having neutral lactase activity has a pH optimum between about pH 6.0 and about pH 8.0. Neutral lactase preparations are usually derived from the cytoplasm of micro-organisms. Their production includes the (large scale) fermentation of the microorganism, followed by isolation of the lactase. The latter requires the disruption of the cell wall in order to release the enzyme from the cytoplasm. Several techniques can be used to obtain cell lysis, including permeabilization of the cell wall by organic solvents such as octanol, sonication or French Pressing. Other enzymes beside lactase are released at the same time from the cytoplasm, including proteases.

Neutral lactase activity may be determined as Neutral Lactase Units (NLU) using o-nitrophenyl-β-D-galactopyranoside (ONPG) as the substrate, according to the procedure described in FCC (fourth ed, July 1996, p 801-802: Lactase (neutral) β-galactosidase activity). One NLU/g may be defined as that quantity of enzyme that liberates 1.30 μM o-nitrophenol/min under assay conditions.

The term “enzyme having neutral lactase activity” includes any auxiliary compounds that may be necessary for the enzyme's catalytic activity, such as, e.g., an appropriate acceptor or cofactor, which may or may not be naturally present in the reaction system.

In one aspect the enzyme having neutral lactase activity has a pH optimum of between about 6.0 and about 8.0.

In one aspect the enzyme having neutral lactase activity is capable of hydrolyzing lactose at a temperature between about 50° C. and about 65° C.

In one aspect, the enzyme having neutral lactase activity is derived from a Lactobacillus spp.

In one aspect, the enzyme having neutral lactase activity is derived from Lactobacillus delbrueckii bulgaricus.

In one aspect, the enzyme having neutral lactase activity has at least about 60% identity to SEQ ID NO:1. In one aspect, the enzyme having neutral lactase activity has at least about 60, 65, 70, 75, 80, 85, 90, 91, 92, 93, 94, 95, 96, 97, 98, 99 or 100% identity to SEQ ID NO:1. In one aspect, the enzyme having neutral lactase activity is that of SEQ ID NO:1, or a lactase active fragment thereof, a homologue thereof or a variant thereof.

“Homologue” means an entity having a certain degree of identity or “homology” with the subject amino acid sequences. In one aspect, the subject amino acid sequence is SEQ ID NO: 1.

A “homologous sequence” includes a polynucleotide or a polypeptide having a certain percent, e.g., 80%, 85%, 90%, 95%, or 99%, of sequence identity with another sequence. Percent identity means that, when aligned, that percentage of bases or amino acid residues are the same when comparing the two sequences. Amino acid sequences are not identical, where an amino acid is substituted, deleted, or added compared to the subject sequence. The percent sequence identity typically is measured with respect to the mature sequence of the subject protein, i.e., following removal of a signal sequence, for example. Typically, homologues will comprise the same active site residues as the subject amino acid sequence. Homologues may retain enzymatic activity of the wild-type or reference enzyme, for example neutral lactase activity.

As used herein, “reference enzymes,” “reference sequence,” “reference polypeptide” mean enzymes and polypeptides from which any of the variant polypeptides are based, e.g., SEQ ID NO: 1. A “reference nucleic acid” means a nucleic acid sequence encoding the reference polypeptide. As used herein, the terms “reference sequence” and “subject sequence” are used interchangeably.

As used here, “Experimental DuPont Lactase” and “LBul” mean the thermostable β-galactosidase from Lactobacillus delbrueckii bulgaricus having the amino acid sequence shown in SEQ ID NO:1.

As used herein, “query sequence” means a foreign sequence, which is aligned with a reference sequence in order to see if it falls within the scope of the present invention. Accordingly, such query sequence can for example be a prior art sequence or a third party sequence.

As used herein, the term “sequence” can either be referring to a polypeptide sequence or a nucleic acid sequence, depending of the context.

As used herein, the terms “polypeptide sequence” and “amino acid sequence” are used interchangeably.

A “variant” or “variants” refers to either polypeptides or nucleic acids. The term “variant” may be used interchangeably with the term “mutant”. Variants include insertions, substitutions, transversions, truncations, and/or inversions at one or more locations in the amino acid or nucleotide sequence, respectively. The phrases “variant polypeptide”, “polypeptide variant”, “polypeptide”, “variant” and “variant enzyme” mean a polypeptide/protein/enzyme that has an amino acid sequence that either has or comprises a selected amino acid sequence of or is modified compared to the selected amino acid sequence, such as SEQ ID NO: 1. Variants may retain enzymatic activity of the wild-type or reference enzyme, for example neutral lactase activity.

As used herein, the term “fragment” is defined herein as a polypeptide having one or more (several) amino acids deleted from the amino and/or carboxyl terminus wherein the fragment has activity.

In one aspect, the term “fragment” is defined herein as a polypeptide having one or more (several) amino acids deleted from the amino and/or carboxyl terminus of the polypeptide of SEQ ID NO: 1, wherein the fragment has neutral lactase activity.

A lactase active fragment of SEQ ID NO: 1 may be any fragment of SEQ ID NO: 1 having neutral lactase activity. For example, a lactase active fragment of SEQ ID NO: 1 may be amino acids 1 to 618; 1 to 713; or 1 to 1002 of SEQ ID NO:1.

### Degree of Sequence Identity

The relatedness between two amino acid sequences or between two nucleotide sequences is described by the parameter “identity”.

In one aspect, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the reference sequence.

In one aspect, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the longest of the two sequences.

In another aspect, the degree of sequence identity between the query sequence and the reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the “alignment length”, where the alignment length is the length of the entire alignment including gaps and overhanging parts of the sequences.

Sequence identity comparisons can be conducted by eye, or more usually, with the aid of readily available sequence comparison programs. These commercially available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalising the insertion of gaps, gap extensions and alignment of non-similar amino acids. The scoring system of the comparison algorithms include:

assignment of a penalty score each time a gap is inserted (gap penalty score),
assignment of a penalty score each time an existing gap is extended with an extra position (extension penalty score),
assignment of high scores upon alignment of identical amino acids, and
assignment of variable scores upon alignment of non-identical amino acids.

Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins DG & Sharp PM (1988), Higgins et al. (1992), Thompson et al. (1994), Larkin et al. (2007). A selection of different alignment tools is available from the ExPASy Proteomics server at www.expasy.org. Another example of software that can perform sequence alignment is BLAST (Basic Local Alignment Search Tool), which is available from the webpage of National Center for Biotechnology Information which can currently be found at www.ncbi.nlm.nih.gov/and which was firstly described in Altschul et al. (1990) J. Mol. Biol. 215; 403-410.

In a preferred aspect of the present invention, the alignment program is performing a global alignment program, which optimizes the alignment over the full-length of the sequences. In a further preferred aspect, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available at www.ebi.ac.uk/Tools/psa/.

EMBOSS Needle performs an optimal global sequence alignment using the Needleman-Wunsch alignment algorithm to find the optimum alignment (including gaps) of two sequences along their entire length.

EMBOSS Stretcher uses a modification of the Needleman-Wunsch algorithm that allows larger sequences to be globally aligned.

In one aspect, the sequences are aligned by a global alignment program and the sequence identity is calculated by identifying the number of exact matches identified by the program divided by the “alignment length”, where the alignment length is the length of the entire alignment including gaps and overhanging parts of the sequences.

In a further aspect, the global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences.

In yet a further aspect, the global alignment program is selected from the group consisting of EMBOSS Needle and EMBOSS stretcher and the sequence identity is calculated by identifying the number of exact matches identified by the program divided by the “alignment length”, where the alignment length is the length of the entire alignment including gaps and overhanging parts of the sequences.

Once the software has produced an alignment, it is possible to calculate % similarity and % sequence identity. The software typically does this as part of the sequence comparison and generates a numerical result.

In one aspect, it is preferred to use the ClustalW software for performing sequence alignments. Preferably, alignment with ClustalW is performed with the following parameters for pairwise alignment:

| | |
|---|---|
| Substitution matrix: | Gonnet 250 |
| Gap open penalty: | 20 |
| Gap extension penalty: | 0.2 |
| Gap end penalty: | None |

ClustalW2 is for example made available on the internet by the European Bioinformatics Institute at the EMBL-EBI webpage www.ebi.ac.uk under tools—sequence analysis—ClustalW2. Currently, the exact address of the ClustalW2 tool is www.ebi.ac.uk/Tools/clustalw2.

In another aspect, it is preferred to use the program Align X in Vector NTI (Invitrogen) for performing sequence alignments. In one aspect, Exp10 has been may be used with default settings:

Gap opening penalty: 10
Gap extension penalty: 0.05
Gap separation penalty range: 8

In a another aspect, the alignment of one amino acid sequence with, or to, another amino acid sequence is determined by the use of the score matrix: blosum62mt2 and the VectorNTI Pair wise alignment settings

| | | |
|---|---|---|
| Settings | K-tuple | 1 |
| | Number of best diagonals | 5 |
| | Window size | 5 |
| | Gap Penalty | 3 |
| | Gap opening Penalty | 10 |
| | Gap extension Penalty | 0, 1 |

In one aspect, the percentage of identity of one amino acid sequence with, or to, another amino acid sequence is determined by the use of Blast with a word size of 3 and with BLOSUM 62 as the substitution matrix.

### Purified Enzymes

In one aspect, the enzyme having neutral lactase activity is purified.

In one aspect the purified enzyme is that of SEQ ID NO:1.

In one aspect, the term “purified” as used herein refers to the enzyme having neutral lactase activity being essentially free from insoluble components from the production organism. In other aspects, the term “purified” also refers to the enzyme having neutral lactase activity being essentially free from insoluble components from the native organism from which it is obtained. In one aspect, the enzyme having neutral lactase activity is also separated from some of the soluble components of the organism and culture medium from which it is derived. The enzyme having neutral lactase activity may be purified (i.e. separated) by one or more of the unit operations: filtration, precipitation, or chromatography.

Accordingly, the enzyme having neutral lactase activity may be purified such that only minor amounts of other proteins are present. The expression “other proteins” relate in particular to other enzymes. The term “purified” as used herein also refers to removal of other components, particularly other proteins and most particularly other enzymes present in the cell of origin of the lactase. The lactase may be “substantially pure”, i.e. free from other components from the organism in which it is produced, i.e., e.g., a host organism for recombinantly produced lactase. In one aspect, the lactase is in an at least 40% (w/w) pure enzyme preparation. In one aspect, the lactase is in at least 50%, 60%, 70%, 80% or 90% pure (w/w) pure enzyme preparation. As used herein, a “substantially pure enzyme” denotes an enzyme preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated.

It is, therefore, preferred that the substantially pure enzyme is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The enzyme of the present invention is preferably in a substantially pure form (i. e., that the preparation is essentially free of other polypeptide material with which it is natively or recombinantly associated). This can be accomplished, for example, by preparing the polypeptide by well-known recombinant methods or by classical purification methods.

Thus, as used herein, the term “substantially free from lipase” means a preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of lipase. Herein, the term “substantially free from” can therefore be seen as being synonymous with the terms “isolated enzyme” and “enzyme in isolated form.” The term “isolated” means that the polypeptide is at least substantially free from at least one other component with which the sequence is naturally associated in nature and as found in nature. In one aspect, “isolated polypeptide” as used herein refers to a polypeptide which is at least 30% pure, at least 40% pure, at least 60% pure, at least 80% pure, at least 90% pure, and at least 95% pure, as determined by SDS-PAGE.

Thus, as used herein, the term “substantially free from p-nitrobenzylesterase” means a preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of p-nitrobenzylesterase. Herein, the term “substantially free from” can therefore be seen as being synonymous with the terms “isolated enzyme” and “enzyme in isolated form.”

Thus, as used herein, the term “substantially free from cellulase” means a preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of cellulase. Herein, the term “substantially free from” can therefore be seen as being synonymous with the terms “isolated polypeptide” and “polypeptide in isolated form.”

As used herein, the term “substantially free from mannanase” means herein a preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of mannanase. Herein, the term “substantially free from” can therefore be seen as being synonymous with the terms “isolated polypeptide” and “polypeptide in isolated form.”

As used herein, the term “substantially free from pectinase” means herein a preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of pectinase. Herein, the term “substantially free from” can therefore be seen as being synonymous with the terms “isolated polypeptide” and “polypeptide in isolated form.”

As used herein, the term “substantially free from amylase” means herein a preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of amylase. Herein, the term “substantially free from” can therefore be seen as being synonymous with the terms “isolated polypeptide” and “polypeptide in isolated form.”

In another aspect the disclosure is directed to a purified enzyme having neutral lactase activity produced by the methods described herein.

In other aspects, the disclosure is related to purified enzymes which are free from lipase side activities, phospholipase side activities, cellulase side activities, pectinase side activities, amylase side activities, protease side activities and/or mannanase side activities. In certain other aspects, the disclosure is related to dairy products comprising purified enzyme produced by the methods of the disclosure.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “heterologous” gene(s) comprise native genes (or ORFs) inserted into a non-native organism and/or chimeric genes inserted into a native or non-native organism.

As used herein, the terms “foreign polynucleotide” or “heterologous polynucleotide” (and variations thereof) are defined as (A) a polynucleotide that is not native to the host cell, (B) a polynucleotide that is native to the host cell, but which polynucleotide has been modified through the use of genetic elements which are not natively associated with the polynucleotide (e.g., heterologous promoters, 5′ UTRs, 3′ UTRs and the like) as isolated from the host cell, or (C) the use of native elements that have been manipulated to function in a manner that does not normally occur in the host cell.

As defined herein, a “heterologous” nucleic acid construct or a “heterologous” nucleic acid sequence has a portion of the sequence which is not native to the cell in which it is expressed.

As used herein, a “transformed cell” includes bacterial cells (e.g., Bacillus cells) which have been transformed by use of recombinant DNA techniques. Transformation generally occurs via the introduction of one or more nucleotide sequences (e.g., polynucleotides) into a cell. The introduced nucleotide sequence(s) may also be a heterologous nucleotide sequence (i. e., a nucleic sequence not endogenous to the cell).

As used herein, the term “nucleic acid construct” refers to a nucleic acid molecule (e.g., a polynucleotide molecule), either single-stranded or double-stranded, which is isolated from a naturally occurring gene or is modified to contain segments of nucleic acids in a manner that would not otherwise exist in nature or is synthetic. The term nucleic acid construct is synonymous with the term “expression cassette” when the nucleic acid construct contains the control sequences required for expression of a coding sequence.

As used herein, the term “control sequences” is defined to include all components, which are necessary or advantageous for the expression of a polynucleotide encoding a polypeptide of the present invention. Each control sequence may be native or foreign to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the term “promoter” is defined as a DNA sequence that binds RNA polymerase and directs the polymerase to the correct downstream transcriptional start site of a polynucleotide encoding a polypeptide. RNA polymerase effectively catalyzes the assembly of messenger RNA complementary to the appropriate DNA strand of the coding region. The term “promoter” will also be understood to include the 5′ non-coding region (between promoter and translation start) for translation after transcription into mRNA, cis-acting transcription control elements such as enhancers, and/or other nucleotide sequences capable of interacting with transcription factors. The promoter can be a wild-type, variant, hybrid, or a consensus promoter.

As used herein, the term “promoter region” is defined as a nucleotide sequence comprising one or more (several) promoter sequences (e.g., a dual promoter, a triple promoter and the like).

As used herein, the term “operably linked” denotes a configuration in which a control sequence (e.g., a promoter sequence) is placed at an appropriate position relative to the coding sequence of the polynucleotide sequence such that the control sequence directs the expression of the coding sequence of a polypeptide. Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG and TGA. The coding sequence may be a DNA, cDNA, synthetic, or recombinant nucleotide sequence.

As used herein, “expression” includes any step involved in the production of a polypeptide of interest (POI) including, but not limited to, transcription, post-transcriptional modification, translation, post-translational modification and secretion.

As used herein, an “expression vector” and “expression construct” are used interchangeably and refer to a linear or circular DNA molecule that comprises a polynucleotide encoding a polypeptide of interest, and is operably linked to additional nucleotides that provide for its expression.

In the present context, “one of the major constituents” means a constituent having a dry matter which constitutes more than 20%, preferably more than 30% or more than 40% of the total dry matter of the dairy product, whereas “the major constituent” means a constituent having a dry matter which constitutes more than 50%, preferably more than 60% or more than 70% of the total dry matter of the dairy product.

### Enzyme Properties

In one aspect, the enzyme having neutral lactase activity is concentrated. In one aspect the enzyme is 1.5, 2, 2.5, 3, 3.5, 4, 4.5, 5, 10, 100, or 1000 times concentrated compared with enzyme prior to concentration. In one aspect the lactase may be concentrated to about 10 to 100 mg/ml.

In one aspect, the activity of the enzyme having neutral lactase activity at 60° C. is at least 50% of the activity at 50° C.

In one aspect the enzyme has a half-life in milk of more than about 4 hours at 55° C., or more than about 1 hour at 58° C.

In one aspect, the enzyme having neutral lactase activity has an optimum temperature of about 60° C.

In one aspect, the enzyme having neutral lactase activity has an optimum pH of about 5.5 to about 8.0. In one aspect, the enzyme having neutral lactase activity has an optimum pH of about 5.7 to about 7.6.

In one aspect the lactase activity of said enzyme having neutral lactase activity at pH 6.0 is at least 50% of its lactase activity at pH 6.5 when measured at 37° C.

In one aspect the galacto-oligosaccharides are DP3+ galacto-oligosaccharides. As such, in one aspect the enzyme having lactase activity converts about 35% or less of lactose in the substrate to DP3+ galacto-oligosaccharides. Preferably, the enzyme having lactase activity converts about 30% or less lactose in the substrate to DP3+ galacto-oligosaccharides, more preferably the enzyme having lactase activity converts about 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.5, 0.4, 0.3, 0.2 or 0.1% or less lactose in the substrate to DP3+ galacto-oligosaccharides

In one aspect, said enzyme having neutral lactase activity has a ratio of lactase:transgalatosylase activity of more than 1:1. In one aspect the contacting is performed under conditions wherein the lactase activity of the enzyme is higher than the transgalactosylating activity, for example 2, 3, 4, 5, 6, 7, 8, 9 or 10 times higher. The ratio of lactase to transgalactosylase activity may be determined, for example, by HPLC analysis, the lactose may be converted into equal amounts of free glucose and free galactose.

“Transgalactosylase” means an enzyme that, among other things, is able to transfer galactose to the hydroxyl groups of D-galactose or D-glucose whereby galacto-oligosaccharides are produced. In one aspect, a transgalactosylase is identified by reaction of the enzyme on lactose in which the amount of galactose generated is less than the amount of glucose generated at any given time.

In the present context, the term “transgalactosylating activity” means the transfer of a galactose moiety to a molecule other than water. The activity can be measured as [glucose]−[galactose] generated at any given time during reaction or by direct quantification of the GOS generated at any given time during the reaction. This measurement may be performed in several ways such as by a HPLC method as shown in the examples. When comparing measurements of transgalactosylating activity, they may have been performed at a given initial lactose concentration, such as e.g. 3, 4, 5, 6, 7, 8, 9 or 10% (w/w).

In one aspect, the enzyme having lactase activity may have a ratio of transgalactosylation activity below 120%.

Ratio of transgalactosylation activity=(Abs420^{+Cellobiose}//Abs420^{-Cellobiose})*100%, where Abs420^{+Cellobiose} is the absorbance read at 420 nm using the described method below including cellobiose in the reaction and Abs420^{-Cellobiose} is the absorbance read at 420 nm using the described method below but without cellobiose in the reaction. The equation above is only valid for dilutions where the absorbance is between 0.5 and 1.0.

### Measuring β-Galactosidase Activity

Enzymatic activity may be measured using the commercially available substrate 2-Nitrophenyl-β-D-Galactopyranoside (ONPG) (Sigma N1127).

ONPG w/o acceptor

| | |
|---|---|
| 100 | mM KPO4 pH 6.0 |
| 12, 3 | mM ONPG |

ONPG Supplemented with Acceptor

| | |
|---|---|
| 100 | mM KPO4 pH6.0 |
| 20 | mM Cellobiose |
| 12, 3 | mM ONPG |

### STOP Solution

10% Na₂CO₃

10 μl dilution series of purified enzyme may be added in wells of a microtiter plates containing 90 μl ONPG-buffer with or without acceptor. Samples may be mixed and incubated for 10 min at 37° C., subsequently 100 μl STOP Solution may be added to each well to terminate reaction. Absorbance measurements may be recorded at 420 nm on a Molecular Device SpectraMax platereader controlled by the Softmax software package.

The ratio of transgalactosylation activity may be calculated as follows Ratio of transgalactosylation activity=(Abs420^{+Cellobiose}/Abs420^{-Cellobiose})*100, for dilutions where the absorbance is between 0.5 and 1.0.

In one aspect, the activity is measured after 15 min. reaction, 30 min. reaction, 60 min. reaction, 90 min. reaction, 120 min. reaction or 180 min. reaction. Thus in one aspect, as an example the relative transgalactosylation activity is measured 15 minutes after addition of enzyme, such as 30 minutes after addition of enzyme, such as 60 minutes after addition of enzyme, such as 90 minutes after addition of enzyme, such as 120 minutes after addition of enzyme or such as 180 minutes after addition of enzyme.

In the present context, the term “ratio of transgalactosylating activity:lactase activity” means ([Glucose]−[Galactose]/[Galactose]).

In the present context, the term [Glucose] means the glucose concentration in % by weight as measured by HPLC.

In the present context, the term [Galactose] means the galactose concentration in % by weight as measured by HPLC.

In the present context, the term “lactose has been transgalactosylated” means that a galactose molecule has been covalently linked to the lactose molecule such as for example covalently linked to any of the free hydroxyl groups in the lactose molecule or as generated by internal transgalatosylation for example forming allolactose.

In one aspect, the enzyme having neutral lactase activity when hydrolysing the lactose in the milk-based substrate or dairy product has a ratio of lactase to transgalactosylase activity of more than 1:1, such as more than 2:1 or more than 3:1. In another aspect, the enzyme treatment is performed under conditions where the lactase activity of the enzyme is higher than the transgalactosylase activity, such as at least two times higher or at least three times higher. The ratio of lactase to transgalactosylase activity in the milk-based substrate may, e.g., be determined by HPLC analysis.

In one aspect, said enzyme having neutral lactase activity has a ratio of lactase activity above 100%. In one aspect, the enzyme having neutral lactase activity has a ratio of lactase activity above 120% such as above 150%, 175% or 200%.

The lactases mentioned herein may be extracellular. They may have a signal sequence at their N-terminus, which is cleaved off during secretion.

The lactases mentioned herein may be derived from any of the sources mentioned herein. The term “derived” means in this context that the enzyme may have been isolated from an organism where it is present natively, i.e. the identity of the amino acid sequence of the enzyme are identical to a native enzyme. The term “derived” also means that the enzymes may have been produced recombinantly in a host organism, the recombinantly produced enzyme having either an identity identical to a native enzyme or having a modified amino acid sequence, e.g. having one or more amino acids which are deleted, inserted and/or substituted, i.e. a recombinantly produced enzyme which is a mutant and/or a fragment of a native amino acid sequence. Within the meaning of a native enzyme are included natural variants. Furthermore, the term “derived” includes enzymes produced synthetically by, e.g., peptide synthesis. The term “derived” also encompasses enzymes which have been modified e.g. by glycosylation, phosphorylation etc., whether in vivo or in vitro. With respect to recombinantly produced enzyme the term “derived from” refers to the identity of the enzyme and not the identity of the host organism in which it is produced recombinantly.

The lactase may be obtained from a microorganism by use of any suitable technique. For instance, a lactase enzyme preparation may be obtained by fermentation of a suitable microorganism and subsequent isolation of a lactase preparation from the resulting fermented broth or microorganism by methods known in the art. The lactase may also be obtained by use of recombinant DNA techniques. Such method normally comprises cultivation of a host cell transformed with a recombinant DNA vector comprising a DNA sequence encoding the lactase in question and the DNA sequence being operationally linked with an appropriate expression signal such that it is capable of expressing the lactase in a culture medium under conditions permitting the expression of the enzyme and recovering the enzyme from the culture. The DNA sequence may also be incorporated into the genome of the host cell. The DNA sequence may be of genomic, cDNA or synthetic origin or any combinations of these, and may be isolated or synthesized in accordance with methods known in the art.

The quality of a lactase can be determined by ratio of side activities to lactase activity. Proteases are known to lead to unwanted side effects, such as milk clotting or off-flavour formation in milk. Off-flavour formation is especially critical in products with a long shelf life and which are stored at room temperatures. One such product is UHT-milk, and off-flavour formation is a known problem for lactose hydrolysed UHT-milk. The UHT-milk is very sensitive to off-flavour formation; when a lactase preparation does not generate off-flavour in UHT-milk, it will usually also not generate off-flavour in other applications. Compounds associated with off-flavour formation in milk, and especially UHT-milk, are related to both proteolysis and Maillard reactions (Valero et al (2001) Food Chem. 72, 51-58). Any proteases present as side activities in lactase preparations potentially enhance the off-flavour formation; it is unclear what levels of proteases are required, but with storage times of several months even very low proteolytic activity could be important. The UHT-milk is very sensitive to off-flavour formation; when a lactase preparation does not generate off-flavour in UHT-milk, other than the off-flavours described (as e.g. described in Valero et al (2001) Food Chem. 72, 51-58) it will usually also not generate off-flavour in other applications. The UHT-application is therefore a good method to evaluate the quality of lactase preparations regarding their off-flavour potential. Since proteases were held at least partly responsible for the off-flavour formation, efforts have focused on reducing protease levels of lactase products.

In one aspect, the enzyme having neutral lactase activity is in the form of an enzyme preparation.

### Enzyme Preparations

In one aspect, the enzyme having neutral lactase activity is in the form of an enzyme preparation which has a reduced level of side activity. A “preparation” is any suitable composition which comprises the enzyme.

The enzyme preparation may reduce the amount of lactose in a substrate by at least about 70%, for example at a temperature of 55° C. or more, or other temperatures as described herein.

In one aspect, the enzyme having neutral lactase activity is in the form of an enzyme preparation which has a reduced level of one or more of the following side activities: lipase activity, protease activity, amylase activity, mannanase activity, pectinase activity, cellulase activity and/or p-nitrobenzylesterase activity.

In one aspect, the enzyme having neutral lactase activity is in the form of an enzyme preparation which has a reduced level of lipase side activity.

In one aspect, the enzyme having neutral lactase activity is in the form of an enzyme preparation which has a reduced level of protease, amylase, mannanase, pectinase, cellulase and/or p-nitrobenzylesterase side activities.

In one aspect, the enzyme preparation has a reduced level of side activity. In one aspect, the enzyme preparation which has a reduced level of one or more of the following side activities: lipase activity, protease activity, amylase activity, mannanase activity, pectinase activity, cellulase activity and/or p-nitrobenzylesterase activity.

In one aspect, the enzyme preparation which has a reduced level of lipase side activity.

In one aspect, the enzyme preparation which has a reduced level of protease, amylase, mannanase, pectinase, cellulase and/or p-nitrobenzylesterase side activities.

The term “reduced level” in connection to the phrases “reduced level of lipase side activity” and “reduced level of protease, amylase, mannanase, pectinase, cellulase and/or p-nitrobenzylesterase side activities” as used herein may refer to a reduced level compared to an enzyme preparation containing an enzyme with neutral lactase activity produced from a different host cell. The reduced level may refer to a reduced level compared to an enzyme preparation produced from an unmodified host cell, i.e. a host cell that has not undergone any of the modifications as described herein. As demonstrated in the present Examples, sensory analysis by a trained panel concluded that milk produced with enzymes produced from a modified host with fewer side activities was preferred. In one aspect the enzyme preparation has improved properties in terms of reduced off-flavour formation. As also described in the Examples, enzyme preparations according to the present invention can lead to improvements in texture, for example in condensed milk.

Without wishing to be bound by theory, the side activities of a lactase (such as lipase activity, protease activity, amylase activity, mannanase activity, pectinase activity, cellulase activity and/or p-nitrobenzylesterase activity) affect the flavour of a milk-based substrate or a dairy product and cause, for example, the dairy product to taste off. Reducing the side activity of a lactase in turn reduces, for example, the levels of off-flavour of the milk-based substrate or dairy product.

The enzyme preparation may advantageously be used in dairy products to hydrolyse lactose without the formation of off-flavour compounds. The lactase may be an intracellular or an extracellular produced lactase. In a preferred aspect, the lactase is intracellular produced lactase. Extracellular lactases have also been described. They are generally recognized as extracellular enzymes because they contain a peptide sequence called leader sequence. This leader sequence is recognized in some way by the cell that produces the enzymes as a signal that the enzyme should be exported out of the cell. During secretion, the leader sequence is usually removed. Extracellular lactases have been described for various species, e.g. Aspergillus oryzae. Crude preparations of extracellular lactases are characterized by the absence of intracellular enzymes and the presence of typical extracellular enzymes like proteases. The type of extracellular enzymes found varies with the organism and are typical for that organism. Due to cell lysis during fermentation or processing, low levels of intracellular enzymes can be found in such extracellular enzyme preparations. Lactase enzymes can thus be classified as extracellular or intracellular based on comparison of their amino acid sequence with those of other known lactases. In principle, an intracellular lactase can be provided with a leader sequence. This could result in excretion of the lactase from the cell into the medium. Crude preparations of such enzymes would be characterized by a lactase, classified as intracellular on basis of its amino acid sequence, in the presence of typical extra-cellular enzymes and absence or low levels of typical intracellular enzymes.

The enzyme or the enzyme preparation may be in any form suited for the use in question, such as, e.g., in the form of a dry powder or granulate, a non-dusting granulate, a liquid, a stabilized liquid, or a protected enzyme.

The enzyme or the enzyme preparation is added in a suitable amount to achieve the desired degree of lactose hydrolysis under the chosen reaction conditions. The enzyme may be added at a concentration of between about 0.1 to 10 g/L milk-based substrate, for example 0.2 to 2 g/L of milk-based substrate. In one aspect the enzyme or enzyme preparation may be added in amount of between about 20 to 300 NLU/g lactose, for example about 24 to 240 NLU/g lactose. per litre milk-based substrate.

As used herein, the terms “lactose reduced” and “lactose free” refer to a reduction in the amount of lactose in a milk-based substrate or a dairy product after contacting said milk-based substrate or dairy product with a lactase.

In one aspect, said amount of lactose is reduced by at least about 70, 75, 80, 85, 90, 95 or 100%.

In one aspect, said lactose is reduced to below 100 ppm or below 1000 ppm.

In some aspect, said lactose is reduced to below 100 ppm at a temperature over 50° C. within 180 minutes. In some aspect, said lactose is reduced to below 100 ppm at a temperature over 50° C. within 120 minutes.

### Milk-Based Substrate

The term “milk” as used herein refers to the lacteal secretion obtained by milking any mammal, such as cows, sheep, goats, buffaloes or camels.

The term “milk-based substrate” as used herein may be any raw and/or processed milk material. In one aspect, said milk-based substrate is selected from solutions/suspensions of any milk or milk like products comprising lactose, such as whole or low fat milk, skim milk, buttermilk, reconstituted milk powder, condensed milk, solutions of dried milk, UHT milk, whey, whey permeate, acid whey, or cream.

In one aspect, the milk-based substrate is milk or an aqueous solution of skim milk powder.

The milk-based substrate may be more concentrated than raw milk.

In one aspect, said milk-based substrate is raw milk which is not pasteurized before contact with said enzyme.

In one aspect, the milk-based substrate comprises about 3 to about 60% lactose. In one aspect, the milk-based substrate comprises about 3 to about 30% lactose. In one aspect, the milk-based substrate comprises about 3 to about 16% lactose.

In one aspect, the milk-based substrate comprises about 3 to about 60% lactose before contacting with an enzyme having neutral lactase activity. In one aspect, the milk-based substrate comprises about 3 to about 30% lactose before contacting with an enzyme having neutral lactase activity. In one aspect, the milk-based substrate comprises about 3 to about 16% lactose before contacting with an enzyme having neutral lactase activity.

In one aspect, the milk-based substrate has a ratio of protein to lactose of at least 0.2, preferably at least 0.3, at least 0.4, at least 0.5, at least 0.6 or, most preferably, at least 0.7.

The milk-based substrate may be homogenized and pasteurized according to methods known in the art.

The term “homogenizing” as used herein means intensive mixing to obtain a soluble suspension or emulsion. It may be performed so as to break up the milk fat into smaller sizes so that it no longer separates from the milk. This may be accomplished by forcing the milk at high pressure through small orifices.

The term “pasteurizing” as used herein means reducing or eliminating the presence of live organisms, such as microorganisms, in the milk-based substrate. In one aspect, pasteurization is attained by maintaining a specified temperature for a specified period of time. The specified temperature is usually attained by heating. The temperature and duration may be selected in order to kill or inactivate certain bacteria, such as harmful bacteria, and/or to inactivate enzymes in the milk. A rapid cooling step may follow.

In one aspect is provided a milk-based substrate having reduced lactose content as described herein. The milk-based substrate with reduced lactose content may be produced by a method as described herein.

### Dairy Product

The present invention encompasses a dairy product obtained or obtainable by any of the methods as described herein.

The term “dairy product” as used herein may be any food product wherein one of the major constituents is milk-based. In one aspect, the major constituent is milk-based. In one aspect, the major constituent is a milk-based substrate which has been treated with an enzyme having neutral lactase activity according to a method of the invention. As used herein, the term “one of the major constituents” means a constituent having a dry matter which constitutes more than 20%, 30% or 40% of the total dry matter of the dairy product. As used herein, the term “the major constituent” means a constituent having a dry matter which constitutes more than 50%, 60% or 70% of the total dry matter of the dairy product.

As described herein, the present invention enables production of a dairy product with reduced lactose content. In one aspect the said amount of lactose is reduced by at least about 70, 75, 80, 85, 90, 91, 92, 93, 94, 95, 96, 97, 98, 99 or 100%, preferably at least about 97%. In one aspect the amount of lactose in the milk-based substrate is reduced by at least about 70, 75, 80, 85, 90, 91, 92, 93, 94, 95, 96, 97, 98, 99 or 100%, preferably at least about 97%. In one aspect the amount of lactose in the dairy product is reduced by at least about 70, 75, 80, 85, 90, 91, 92, 93, 94, 95, 96, 97, 98, 99 or 100%, preferably at least about 97%, compared to the milk-based substrate.

In one aspect the dairy product produced by the methods described herein contains below 100 ppm lactose. In one aspect the dairy product produced by the methods described herein contains below 1000 ppm lactose.

In one aspect the lactose is reduced to below 100 ppm at a temperature over 50° C. within about 180 minutes, preferably within about 170, 160, 150, 140, 130, 120, 110, 100 or 90 minutes. In one aspect about 35 to about 100 mg enzyme/l milk may be used. In one aspect about 0.75 to about 2.2 mg enzyme/g lactose may be used.

A dairy product as described herein may be, e.g., skim milk, low fat milk, whole milk, cream, UHT milk, milk having an extended shelf life, a fermented milk product, cheese, yoghurt, butter, dairy spread, butter milk, acidified milk drink, sour cream, whey based drink, condensed milk, dulce de leche, a flavoured milk drink, sweetened condensed milk, milk powder, reconstituted dairy products, ice-cream, Ryazhenka, pudding, desserts or milk-shakes. A dairy product may be manufactured by any method known in the art.

In one aspect, said dairy product is selected from the group consisting of skim milk, low fat milk, whole milk, cream, UHT milk, milk having an extended shelf life, a fermented milk product, cheese, yoghurt, butter, dairy spread, butter milk, acidified milk drink, sour cream, whey based drink, condensed milk, dulce de leche, a flavoured milk drink, sweetened condensed milk, milk powder, reconstituted dairy products, ice-cream, Ryazhenka, pudding, desserts and milk-shakes. In one aspect said dairy product is condensed milk, ice cream or milk-shake. In one aspect said dairy product is milk powder or whey powder.

A “fermented dairy product” is to be understood as any dairy product wherein any type of fermentation forms part of the production process. Examples of fermented dairy products are products like yoghurt, buttermilk, creme fraiche, quark and fromage frais. Another example of a fermented dairy product is cheese. A fermented dairy product may be produced by any method known in the art.

The term “fermentation” means the conversion of carbohydrates into alcohols or acids through the action of a microorganism such as a starter culture. In one aspect, fermentation comprises conversion of lactose to lactic acid.

In the present context, “microorganism” may include any bacterium or fungus being able to ferment a milk substrate.

A dairy product may additionally comprise non-milk components, e.g. vegetable components such as, e.g., vegetable oil, vegetable protein, and/or vegetable carbohydrates. Dairy products may also comprise further additives such as, e.g., enzymes, flavouring agents, microbial cultures such as probiotic cultures, salts, sweeteners, sugars, acids, fruit, fruit juices, or any other component known in the art as a component of, or additive to, a dairy product.

### Method of Reducing Lactose

In one aspect, the method comprises contacting a milk-based substrate with an enzyme having neutral lactase activity at a temperature of about 50° C. to about 90° C., preferably about 50° C. to about 65° C., more preferably about 55° C. to about 60° C.

In one aspect, the method comprises contacting a milk-based substrate with an enzyme having neutral lactase activity as described herein at a temperature selected from about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 and 90° C.

In one aspect, the method comprises contacting a milk-based substrate with an enzyme having neutral lactase activity at a temperature of about 55 or 58° C.

In one aspect the temperature is about 55° C. For example, when the dairy product is condensed milk the temperature may be about 55° C. In one aspect the temperature may be about 63° C., for example for a time of about 30 minutes.

In one aspect said contacting is performed for a period of minutes, or a period of hours, for example about 1 to 60 minutes or about 1 to 5 hours. In one aspect said contacting is performed for between about 10 minutes and about 4 hours. In one aspect said contacting is performed between about 10 minutes and about 3 hours. In one aspect said contacting is performed between about 10 minutes and about 2 hours. In one aspect said contacting is performed between about 10 minutes and about 1 hour.

In one aspect, said enzyme having neutral lactase activity is added to the milk-based substrate at a concentration of about 20 to about 300 NLU/g lactose, for example about 24 to about 240 NLU/g lactose, more preferably about 60 to about 70 NLU/g lactose.

The invention encompasses method for producing a lactose reduced or lactose free milk shake, ice cream, reconstituted milk product, desserts, pudding, condensed milk, sweetened condensed milk, Ryazhenka, Dulce de Leche or milk based powder, said method comprising contacting a milk-based substrate with a lactase at a temperature of about 50 to about 65° C.

In one aspect, said method includes a pasteurization step. “Pasteurizing” as used herein means reducing or eliminating the presence of live organisms, such as microorganisms, in the milk-based substrate. Preferably, pasteurization is attained by maintaining a specified temperature for a specified period of time. The specified temperature is usually attained by heating. The temperature and duration may be selected in order to kill or inactivate certain bacteria, such as harmful bacteria, and/or to inactivate enzymes in the milk. A rapid cooling step may follow.

In one aspect, said method comprises adding said enzyme having neutral lactase activity after pasteurization and/or homogenization of the milk-based substrate. The contacting may be during the cooling to fermentation temperature. In one aspect, said method comprises adding said enzyme having neutral lactase activity before or during pasteurization and/or homogenization. In one aspect the pasteurizing may take place at about 72° C. In one aspect the pasteurizing may take place for a period of between 15 and 30 seconds, for example between 10 and 20 seconds, preferably about 15 seconds. In one aspect the pasteurizing may take place for a period of about 15, 16, 17, 18, 19, 20, 21, 22, 23, 24, 25, 26, 27, 28, 29 or 30 seconds.

In one aspect about 10, 15, 20, 25, 30 or 35%, preferably about 20 to 30%, more preferably about 20%, lactase activity may remain in the milk-based substrate after pasteurization.

In one aspect the milk-based substrate does not undergo pasteurization. In one aspect the substrate if raw milk that is not pasteurized prior to contact with the enzyme as described herein.

“Homogenizing” as used herein means intensive mixing to obtain a soluble suspension or emulsion. It may be performed so as to break up the milk fat into smaller sizes so that it no longer separates from the milk. This may be accomplished by forcing the milk at high pressure through small orifices.

In one aspect the contacting may be during the evaporation process. In one aspect the contacting may be during the condensing process. In one aspect the contacting takes place during pasteurization.

### Host Cell

The terms “host”, “host cell” and “cell” may be used interchangeably herein.

In one aspect, the host is a bacterial expression host.

In one aspect, the bacterial expression host is capable of expressing an enzyme having neutral lactase activity as described herein.

In one aspect the host comprises a genetic modification which reduces or eliminates lipase activity. In one aspect, the bacterial expression host capable of expressing an enzyme having neutral lactase activity comprises a genetic modification which reduces or eliminates lipase A activity.

In one aspect, the genetic modification comprises a deletion, disruption or down-regulation of a gene encoding a lipase. In one aspect, the genetic modification comprises a deletion, disruption or down-regulation of a gene encoding a Lipase A polypeptide, for example as set out in SEQ ID NO:2.

In one aspect, the host comprises a genetic modification which reduces or eliminates one or more enzyme activity selected from: protease, amylase, mannanase, pectinase, cellulase and p-nitrobenzylestaerase activities.

In one aspect, the host comprises a genetic modification which reduces or eliminates lipase, preferably lipase A, protease, amylase, mannanase, pectinase, cellulase and p-nitrobenzylestaerase activities.

In one aspect the host comprises a genetic modification which reduces or eliminates cellulase activity.

In one aspect, the bacterial expression host is a Bacillus sp cell.

In one aspect the bacterial expression host is selected from the group consisting of B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B. akibai, B. clarkii, B. pseudofirmus, B. lehensis, B. megaterium, B. coagulans, B. circulans, B. gibsonii, and B. thuringiensis. In one aspect, the bacterial expression host is a Bacillus subtilis.

In one aspect, the bacterial expression host comprises a nucleic acid molecule as described herein.

In one aspect, said host expresses an enzyme having neutral lactase activity as described herein.

Modification, downregulation, or inactivation of a nucleic acid molecule encoding an enzyme having neutral lactase activity may be obtained via RNA interference (RNAi) techniques {FEMS Microb. Lett. 237:317-324, 2004). More specifically, expression of the gene by a filamentous fungal cell may be reduced or eliminated by cloning identical sense and antisense portions of the nucleotide sequence, which expression is to be affected, behind each other with a nucleotide spacer in between, inserting into an expression vector, and introducing the expression vector into the cell where double-stranded RNA (dsRNA) may be transcribed and then processed to shorter siRNA that is able to hybridize to target mRNA. After dsRNA is transcribed, formation of small (21-23) nucleotide siRNA fragments will lead to a targeted degradation of the mRNA, which is to be affected. The elimination of the specific mRNA can be to various extents. The RNA interference techniques described in WO 2005/05672 and WO 2005/026356 may be used for modification, downregulation, or inactivation of a nucleic acid molecule encoding an enzyme having neutral lactase activity.

One aspect is directed to genetically modified Bacillus host cells capable of expressing/producing one or more lactases of the disclosure. For example, International PCT Publication No. WO2016/071504 discloses recombinant Bacillus subtilis host cells transformed with polynucleotide constructs (e.g., expression constructs) encoding various β-galactosidases. Thus, Bacillus host cells are well known in the art as expression hosts and are particularly suitable host cells for expressing/producing lactases of the disclosure.

In certain aspects, the disclosure is therefore directed to methods for genetically modifying Bacillus cells, wherein the modification comprises (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) a gene down-regulation, (f) site specific mutagenesis and/or (g) random mutagenesis.

Thus, a modified Bacillus cell of the disclosure is constructed by reducing or eliminating the expression of gene encoding any of the side activities described herein, using methods well known in the art, for example, insertions, disruptions, replacements, deletions, truncations, substitutions, frame shift mutations and the like. The portion of the gene to be modified or inactivated may be, for example, the coding region or a regulatory/control element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other aspects a modified Bacillus cell is constructed by gene deletion to eliminate or reduce the expression of the gene. Gene deletion techniques enable the partial or complete removal of the gene(s), thereby eliminating their expression, or expressing a non-functional (or reduced activity) protein product. In such methods, the deletion of the gene may be accomplished by homologous recombination using a plasmid that has been constructed to contiguously contain the 5′ and 3′ regions flanking the gene. The contiguous 5′ and 3′ regions may be introduced into a Bacillus cell, for example, on a temperature-sensitive plasmid, such as pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, e.g., Perego, 1993). Thus, a person of skill in the art (e.g., by reference to the >-nitrobenzylesterase gene's (nucleic acid) sequence and the encoded protein sequence thereof), may readily identify nucleotide regions in the gene's coding sequence and/or the gene's non-coding sequence suitable for complete or partial deletion.

In other aspects, a modified Bacillus cell of the disclosure is constructed by introducing, substituting, or removing one or more nucleotides in the gene or a regulatory element required for the gene transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art (e.g., see, Botstein and Shortle, 1985; Lo et al, 1985; Higuchi et al, 1988; Shimada, 1996; Ho et al, 1989; Horton et al, 1989 and Sarkar and Sommer, 1990). Thus, in certain aspects, a >-nitrobenzylesterase gene of the disclosure is inactivated by complete or partial deletion.

In another aspect, a modified Bacillus cell is constructed by the process of gene conversion (e.g., see Iglesias and Trautner, 1983). For example, in the gene conversion method, a nucleic acid sequence corresponding to the gene is mutagenized in vitro to produce a defective nucleic acid sequence, which is then transformed into the parental Bacillus cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene (Perego, 1993). Alternatively, the defective nucleic acid sequence may contain an insertion, substitution, or deletion of one or more nucleotides of the gene, as described below.

In other aspects, a modified Bacillus cell is constructed by established anti-sense techniques using a nucleotide sequence complementary to the nucleic acid sequence of the gene (Parish and Stoker, 1997). More specifically, expression of the gene by a Bacillus cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated from the gene is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other aspects, a modified Bacillus cell is produced/constructed via CRISPR-Cas9 editing. For example, a gene encoding any of the side activities mentioned herein can be disrupted (or deleted or down-regulated) by means of nucleic acid guided endonucleases, that find their target DNA by binding either a guide RNA (e.g., Cas9) and Cpf 1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Bacillus cell and a terminator active in Bacillus cell, thereby creating a Bacillus Cas9 expression cassette. Likewise, one or more target sites unique to the gene are readily identified by a person skilled in the art. For example, to build a DNA construct encoding a gRNA-directed to a target site within the gene of interest, the variable targeting (VT) domain will comprise nucleotides of the target site which are 5′ of the (PAM) proto-spacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Bacillus expression cassette for the gRNA is created by operably linking the DNA encoding the gRNA to a promoter active in Bacillus cells and a terminator active in Bacillus cells.

In yet other aspects, a modified Bacillus cell is constructed by random or specific mutagenesis using methods well known in the art, including, but not limited to, chemical mutagenesis (see, e.g., Hopwood, 1970) and transposition (see, e.g., Youngman et al., 1983). Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

In other aspects, a modified Bacillus cell comprises a disruption of an endogenous gene, wherein the polynucleotide disruption cassette comprises a marker gene.

PCT Publication No. WO2003/083125 discloses methods for modifying Bacillus cells, such as the creation of Bacillus deletion strains and DNA constructs using PCR fusion to bypass E. coli.

PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E. coli and Bacillus sp.) (e.g., Ferrari et al, 1989; Saunders et al, 1984; Hoch et al, 1967; Mann et al, 1986; Holubova, 1985; Chang et al, 1979; Vorobjeva et al, 1980; Smith et al, 1986; Fisher et. al, 1981 and McDonald, 1984). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some aspects, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional aspects, DNA constructs are co-transformed with a plasmid without being inserted into the plasmid. In further aspects, a selective marker is deleted or substantially excised from the modified Bacillus strain by methods known in the art (e.g., Stahl et al, 1984 and Palmeros et al., 2000). In some aspects, resolution of the vector from a host chromosome leaves the flanking regions in the chromosome, while removing the indigenous chromosomal region.

In one aspect is provided a method for expressing an enzyme as described herein, comprising providing a host cell as described herein and expressing the enzyme from the cell, and optionally purifying and/or concentrating the enzyme. In one aspect is provided an enzyme produced by such a method. Also provided is a dairy product comprising the expression host as described herein, or the enzyme described herein which h has been produced in the expression host.

### Nucleic Acids and Vectors

In one aspect, the present invention relates to isolated enzymes (polypeptides) having neutral lactase activity as stated above which are encoded by polynucleotides which hybridize under very low stringency conditions, preferably low stringency conditions, more preferably medium stringency conditions, more preferably medium-high stringency conditions, even more preferably high stringency conditions, and most preferably very high stringency conditions with i) a nucleic acid sequence encoding the mature polypeptide of SEQ ID NO: 1; ii) the cDNA sequence of i) or iii) the complementary strand of i) or ii), (J. Sambrook, E. F. Fritsch, and T. Maniatis, 1989, Molecular Cloning. A Laboratory Manual, 2d edition, Cold Spring Harbor, N.Y.). A subsequence of the nucleic acid sequence contains at least 100 contiguous nucleotides or preferably at least 200 contiguous nucleotides. Moreover, the subsequence may encode a polypeptide fragment which has neutral lactase activity.

The nucleotide sequence as described herein or a subsequence thereof, as well as the amino acid sequence of SEQ ID NO: 1 or a fragment thereof, may be used to design a nucleic acid probe to identify and clone DNA encoding polypeptides having neutral lactase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes which are at least 600 nucleotides, at least preferably at least 700 nucleotides, more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA library prepared from such other organisms may, therefore, be screened for DNA which hybridizes with the probes described above and which encodes a polypeptide having lactase activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with a nucleic acid sequence as described herein or a subsequence thereof, the carrier material is used in a Southern blot

For purposes of the present invention, hybridization indicates that the nucleotide sequence hybridizes to a labelled nucleic acid probe corresponding to the nucleotide sequence described herein, its complementary strand, or a subsequence thereof, under very low to very high stringency conditions. Molecules to which the nucleic acid probe hybridizes under these conditions can be detected using X-ray film.

In another preferred aspect, the nucleic acid probe is the mature polypeptide coding region of the nucleic acid sequence.

For long probes of at least 100 nucleotides in length, very low to very high stringency conditions are defined as prehybridization and hybridization at 42° C. in 5×SSPE, 0.3% SDS, 200 g/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

In a particular aspect, the wash is conducted using 0.2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency). In another particular aspect, the wash is conducted using 0.1×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

Under salt-containing hybridization conditions, the effective Tₘ is what controls the degree of identity required between the probe and the filter bound DNA for successful hybridization. The effective Tₘ may be determined using the formula below to determine the degree of identity required for two DNAs to hybridize under various stringency conditions.

Effective Tₘ=81.5+16.6(log M[Na⁺])+0.41(% G+C)−0.72(% formamide

For medium stringency, the formamide is 35% and the Na⁺ concentration for 5×SSPE is 0.75 M.

Another relevant relationship is that a 1% mismatch of two DNAs lowers the Tₘ by 1.4° C. To determine the degree of identity required for two DNAs to hybridize under medium stringency conditions at 42° C., the following formula is used:

% Homology=100−[(Effective Tₘ−Hybridization Temperature)/1.4]

(See www.ndsu.nodak.edu/instruct/mcclean/plsc731/dna/dna6.htm)

The variant nucleic acids include a polynucleotide having a certain percent, e.g., 60%, 65%, 70%, 75%, 80%, 85%, 90%, 95%, or 99%, of sequence identity with the nucleic acid encoding SEQ ID NO: 1. In one aspect, a nucleic acid capable of encoding an enzyme (polypeptide) as disclosed herein, is provided. In a further aspect, nucleic acid has a nucleic acid sequence which is at least 60%, such as at least 65%, such as at least 70%, such as at least 75%, such as at least 80%, such as at least 85%, such as at least 90%, such as at least 95%, such as at least 99% identical to the nucleic acid capable of encoding an enzyme (polypeptide) as disclosed herein.

In one aspect, a plasmid comprising a nucleic acid as described herein, is provided.

In one aspect, an expression vector comprising a nucleic acid as described herein, or capable of expressing a polypeptide as described herein, is provided.

A nucleic acid complementary to a nucleic acid encoding any of the polypeptide variants as defined herein set forth herein is provided. Additionally, a nucleic acid capable of hybridizing to the complement is provided. In another aspect, the sequence for use in the methods and compositions described here is a synthetic sequence. It includes, but is not limited to, sequences made with optimal codon usage for expression in host organisms, such as yeast.

The polypeptide variants as provided herein may be produced synthetically or through recombinant expression in a host cell, according to procedures well known in the art. In one aspect, the herein disclosed polypeptide(s) is recombinant polypeptide(s). The expressed polypeptide variant as defined herein optionally is isolated prior to use.

In another aspect, the polypeptide variant as defined herein is purified following expression. Methods of genetic modification and recombinant production of polypeptide variants are described, for example, in U.S. Pat. Nos. 7,371,552, 7,166,453; 6,890,572; and 6,667,065; and U.S. Published Application Nos. 2007/0141693; 2007/0072270; 2007/0020731; 2007/0020727; 2006/0073583; 2006/0019347; 2006/0018997; 2006/0008890; 2006/0008888; and 2005/0137111. The relevant teachings of these disclosures, including polypeptide-encoding polynucleotide sequences, primers, vectors, selection methods, host cells, purification and reconstitution of expressed polypeptide variants, and characterization of polypeptide variants as defined herein, including useful buffers, pH ranges, Ca²⁺ concentrations, substrate concentrations and enzyme concentrations for enzymatic assays, are herein incorporated by reference.

A nucleic acid sequence is provided encoding the protein of SEQ ID NO: 1 or a nucleic acid sequence having at least about 66%, 68%, 70%, 72%, 74%, 78%, 80%, 85%, 90%, 95%, 96%, 97%, 98%, 99% or 100% sequence identity with a nucleic acid encoding the enzyme of SEQ ID NO: 1. In one aspect, the nucleic acid sequence has at least about 60%, 66%, 68%, 70%, 72%, 74%, 78%, 80%, 85%, 90%, 95%, 96%, 97%, 98% or 99% sequence identity to the nucleic acid encoding the enzyme of SEQ ID NO: 1.

In one aspect, the invention relates to a vector comprising a polynucleotide. In one aspect, a bacterial cell comprises the vector. In some aspects, a DNA construct comprising a nucleic acid encoding a variant is transferred to a host cell in an expression vector that comprises regulatory sequences operably linked to an encoding sequence. The vector may be any vector that can be integrated into a fungal host cell genome and replicated when introduced into the host cell. The FGSC Catalogue of Strains, University of Missouri, lists suitable vectors. Additional examples of suitable expression and/or integration vectors are provided in Sambrook et al., Molecular Cloning: A Laboratory Manual, 3^{rd} ed., Cold Spring Harbor Laboratory Press, Cold Spring Harbor, N.Y. (2001); Bennett et al., More Gene Manipulations in Fungi, Academic Press, San Diego (1991), pp. 396-428; and U.S. Pat. No. 5,874,276. Exemplary vectors include pFB6, pBR322, PUC18, pUC100 and pENTR/D, pDON™201, pDONR™221, pENTR™, pGEM®3Z and pGEM®4Z. Exemplary for use in bacterial cells include pBR322 and pUC19, which permit replication in E. coli, and pE194, for example, which permits replication in Bacillus.

In some aspects, a nucleic acid encoding a variant is operably linked to a suitable promoter, which allows transcription in the host cell. The promoter may be derived from genes encoding proteins either homologous or heterologous to the host cell. Suitable non-limiting examples of promoters include cbh1, cbh2, egl1, and egl2 promoters. In one aspect, the promoter is one that is native to the host cell. For example, when P. saccharophila is the host, the promoter is a native P. saccharophila promoter. An “inducible promoter” is a promoter that is active under environmental or developmental regulation. In another aspect, the promoter is one that is heterologous to the host cell.

In some aspects, the coding sequence is operably linked to a DNA sequence encoding a signal sequence. In another aspect, a representative signal peptide may be the native signal sequence of the Bacillus subtilis aprE precursor. In other aspects, the DNA encoding the signal sequence is replaced with a nucleotide sequence encoding a signal sequence from other extra-cellular Bacillus subtilis pre-cursors. In one aspect, the polynucleotide that encodes the signal sequence is immediately upstream and in-frame of the polynucleotide that encodes the polypeptide. The signal sequence may be selected from the same species as the host cell.

In additional aspects, a signal sequence and a promoter sequence comprising a DNA construct or vector to be introduced into a fungal host cell are derived from the same source. In some aspects, the expression vector also includes a termination sequence. In one aspect, the termination sequence and the promoter sequence are derived from the same source. In another aspect, the termination sequence is homologous to the host cell.

In some aspects, an expression vector includes a selectable marker. Examples of suitable selectable markers include those that confer resistance to antimicrobial agents, e.g., hygromycin or phleomycin. Nutritional selective markers also are suitable and include amdS, argB, and pyr4. In one aspect, the selective marker is the amdS gene, which encodes the enzyme acetamidase; it allows transformed cells to grow on acetamide as a nitrogen source. The use of an A. nidulans amdS gene as a selective marker is described in Kelley et al., EMBO J. 4: 475-479 (1985) and Penttila et al., Gene 61: 155-164 (1987).

A suitable expression vector comprising a DNA construct with a polynucleotide encoding a variant may be any vector that is capable of replicating autonomously in a given host organism or integrating into the DNA of the host. In some aspects, the expression vector is a plasmid. In some aspects, two types of expression vectors for obtaining expression of genes are contemplated. The first expression vector comprises DNA sequences in which the promoter, coding region, and terminator all originate from the gene to be expressed. In some aspects, gene truncation is obtained by deleting undesired DNA sequences to leave the domain to be expressed under control of its own transcriptional and translational regulatory sequences. The second type of expression vector is preassembled and contains sequences required for high-level transcription and a selectable marker. In some aspects, the coding region for a gene or part thereof is inserted into this general-purpose expression vector, such that it is under the transcriptional control of the expression construct promoter and terminator sequences. In some aspects, genes or part thereof are inserted downstream of the strong cbh1 promoter.

In a further aspect, a method of expressing a polypeptide as described herein comprises obtaining a host cell or a cell as described herein and expressing the polypeptide from the cell or host cell, and optionally purifying the polypeptide.

An expression characteristic means an altered level of expression of the variant, when the variant is produced in a particular host cell. Expression generally relates to the amount of active variant that is recoverable from a fermentation broth using standard techniques known in this art over a given amount of time. Expression also can relate to the amount or rate of variant produced within the host cell or secreted by the host cell. Expression also can relate to the rate of translation of the mRNA encoding the variant polypeptide.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See. e.g., Ausubel et al. (1987), supra, chapter 9; Sambrook et al. (2001), supra; and Campbell et al., Curr. Genet. 16: 53-56 (1989). The expression of heterologous protein in Trichoderma is described, for example, in U.S. Pat. Nos. 6,022,725; 6,268,328; Harkki et al., Enzyme Microb. Technol. 13: 227-233 (1991); Harkki et al., BioTechnol. 7: 596-603 (1989); EP 244,234; and EP 215,594. In one aspect, genetically stable transformants are constructed with vector systems whereby the nucleic acid encoding a variant is stably integrated into a host cell chromosome. Transformants are then purified by known techniques.

In one non-limiting example, stable transformants including an amdS marker are distinguished from unstable transformants by their faster growth rate and the formation of circular colonies with a smooth, rather than ragged outline on solid culture medium containing acetamide. Additionally, in some cases a further test of stability is conducted by growing the transformants on solid non-selective medium, e.g., a medium that lacks acetamide, harvesting spores from this culture medium and determining the percentage of these spores that subsequently germinate and grow on selective medium containing acetamide. Other methods known in the art may be used to select transformants.

To evaluate the expression of a variant in a host cell, assays can measure the expressed protein, corresponding mRNA, or neutral lactase activity. For example, suitable assays include Northern and Southern blotting, RT-PCR (reverse transcriptase polymerase chain reaction), and in situ hybridization, using an appropriately labeled hybridizing probe. Suitable assays also include measuring activity in a sample. Suitable assays of the activity of the variant include, but are not limited to, ONPG based assays or determining glucose in reaction mixtures such for example described in the methods and examples herein.

In general, a variant produced in cell culture is secreted into the medium and may be purified or isolated, e.g., by removing unwanted components from the cell culture medium. In some cases, a variant may be recovered from a cell lysate. In such cases, the enzyme is purified from the cells in which it was produced using techniques routinely employed by those of skill in the art. Examples include, but are not limited to, affinity chromatography, ion-exchange chromatographic methods, including high resolution ion-exchange, hydrophobic interaction chromatography, two-phase partitioning, ethanol precipitation, reverse phase HPLC, chromatography on silica or on a cation-exchange resin, such as DEAE, chromatofocusing, SDS-PAGE, ammonium sulfate precipitation, and gel filtration using Sephadex G-75, for example. Depending on the intended use the herein disclosed polypeptide(s) may for example be either freeze-dried or prepared in a solution. In one aspect, the herein disclosed polypeptide(s) is freeze-dried form. In another aspect, the herein disclosed polypeptide(s) is in solution. The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the enzymes or enzyme preparations of the invention.

In one aspect, the amount of milk sugar in milk based substrate, for example fresh, low fat, or skim milk, typically having about 4.7 to about 5.0% lactose, or condensed or concentrated milk having from about 6.0 to about 65% lactose may be reduced by adding a lactase capable of converting lactose to TGOS, i.e., having transgalactosylating activity. Here, “milk sugar” is defined as lactose (DP2) and monosaccharides (DP1) derived from the hydrolysis of lactose, including glucose and galactose and other DP2 molecules like allolactose and DP2 GOS. DP3+ fibers are not sugar. Hence, treatment of a milk based substrate having a given amount of lactose with a lactase having transgalactosylase activity can result in sugar reduction where lactose is converted into GOS fibers (DP3+).

According to this aspect of the present invention, the milk based substrate preferably contains 6-9% lactose, 9-20% lactose, 20-40% lactose or 40-65% lactose. Milk sugar in the milk based substrate is preferably reduced more than 20%, more than 25%, more than 30% and more than 40%.

In one aspect, disclosed herein is a method for producing a food product by treating a substrate comprising lactose with an enzyme as described herein.

In one aspect, disclosed herein is a method for producing a dairy product by treating a milk-based substrate comprising lactose with an enzyme as described herein.

The enzyme preparation, such as in the form of a food ingredient prepared according to the present invention, may be in the form of a solution or as a solid—depending on the use and/or the mode of application and/or the mode of administration. The solid form can be either as a dried enzyme powder or as a granulated enzyme.

Examples of dry enzyme formulations include spray dried products, mixer granulation products, layered products such as fluid bed granules, extruded or pelletized granules, prilled products, and lyophilyzed products.

The enzyme preparation, such as in the form of a food ingredient prepared according to the present invention, may be in the form of a solution or as a solid—depending on the use and/or the mode of application and/or the mode of administration. The solid form can be either as a dried enzyme powder or as a granulated enzyme.

In one aspect, a composition preferably a food composition, more preferably a dairy product comprising a host cell or an enzyme as described herein, is provided.

Furthermore, disclosed herein is a composition or preparation comprising at least 5%, such as e.g. 10%, 15%, 20%, 25%, 30%, 35%, 40%, 45%, 50% w/w of one or more enzyme as disclosed herein based on the total amount of enzyme in the composition having at least 70%, e.g. such as 72%, 74%, 74%, 78%, 80%, 82%, 84%, 86%, 88%, 90% sequence identity with SEQ ID NO: 1. This may be evaluated by using the following techniques know to a person skilled in the art. The samples to be evaluated are subjected to SDS-PAGE and visualized using a dye appropriate for protein quantification, such as for example the Bio-Rad Criterion system. The gel is then scanned using appropriate densiometic scanner such as for example the Bio-Rad Criterion system and the resulting picture is ensured to be in the dynamic range. The bands corresponding to any variant/fragment are quantified and the percentage of the polypeptides are calculated as: Percentage of polypeptide in question=polypeptide in question/(sum of all polypeptides exhibiting transgalactosylating activity)*100. The total number of polypeptides variants/fragments in the composition can be determined by detecting fragment by western blotting using a polyclonal antibody by methods know to a person skilled in the art.

In one aspect the invention provides an enzyme preparation comprising an enzyme according to the invention, an enzyme carrier and optionally a stabilizer and/or a preservative.

In yet a further aspect of the invention, the enzyme carrier is selected from the group consisting of glycerol or water.

In a further aspect, the preparation/composition comprises a stabilizer. In one aspect, the stabilizer is selected from the group consisting of inorganic salts, polyols, sugars and combinations thereof. In one aspect, the stabilizer is an inorganic salt such as potassium chloride. In another aspect, the polyol is glycerol, propylene glycol, or sorbitol. In yet another aspect, the sugar is a small-molecule carbohydrate, in particular any of several sweet-tasting ones such as glucose, galactose, fructose and saccharose.

In yet at further aspect, the preparation comprises a preservative. In one aspect, the preservative is methyl paraben, propyl paraben, benzoate, sorbate or other food approved preservatives or a mixture thereof.

The method of the invention may be practiced with immobilized enzymes, e.g. an immobilized lactase or other galactooligosaccharide producing enzymes. The enzyme can be immobilized on any organic or inorganic support. Exemplary inorganic supports include alumina, celite, Dowex-1-chloride, glass beads and silica gel. Exemplary organic supports include DEAE-cellulose, alginate hydrogels or alginate beads or equivalents. In various aspects of the invention, immobilization of the lactase can be optimized by physical adsorption on to the inorganic support. Enzymes used to practice the invention can be immobilized in different media, including water, Tris-HCl buffer and phosphate buffered solution. The enzyme can be immobilized to any type of substrate, e.g. filters, fibers, columns, beads, colloids, gels, hydrogels, meshes and the like.

In one aspect, a method for producing a dairy product by treating a milk-based substrate comprising lactose with an enzyme as described herein is provided. In one aspect, a method is provided, wherein the treatment with an enzyme as described herein takes place at an optimal temperature for the activity of the enzyme, or for example at the higher temperatures described herein.

In a further aspect, the polypeptide is added to the milk-based substrate at a concentration of 0.01-1000 ppm. In yet a further aspect, the polypeptide is added to the milk-based substrate at a concentration of 0.1-100 ppm. In a further aspect, the polypeptide is added to the milk-based substrate at a concentration of 1 to 60 ppm, for example 1 to 55, 1 to 50, 1 to 45, 1 to 40, 1 to 35, 1 to 30, 1 to 25, 1 to 20, 1 to 15, 1 to 10 or 1 to 5 ppm, preferably 1 to 10 ppm.

In one aspect, a method further comprising fermenting a substrate such as a dairy product with a microorganism, is provided. In a further aspect, the dairy product is yogurt. In a further aspect, the treatment with the enzyme and the microorganism is performed essentially at the same time. In one aspect, the polypeptide and the microorganism are added to the milk-based substrate essentially at the same time.

In one aspect, a dairy product comprising a cell or an enzyme as described herein, is provided. In one aspect, the enzyme as defined herein is added in a concentration of 0.01-1000 ppm. In one aspect, a dairy product comprising an inactivated enzyme as defined herein, is provided. In one aspect, a dairy product comprising an inactivated enzyme as defined herein in a concentration of 0.01-1000 ppm, is provided. In one aspect, a dairy product comprising a cell as defined herein, is provided.

A dairy product as described herein may be, skim milk, low fat milk, whole milk, cream, UHT milk, milk having an extended shelf life, a fermented milk product, cheese, yoghurt, butter, dairy spread, butter milk, acidified milk drink, sour cream, whey based drink, condensed milk, dulce de leche, a flavoured milk drink, sweetened condensed milk, milk powder, reconstituted dairy products, ice-cream, Ryazhenka, pudding, desserts and milk-shakes. A dairy product may be manufactured by any method known in the art.

A dairy product may additionally comprise non-milk components, e.g. vegetable components such as, e.g., vegetable oil, vegetable protein, and/or vegetable carbohydrates. Dairy products may also comprise further additives such as, e.g., enzymes, flavouring agents, microbial cultures such as probiotic cultures, salts, sweeteners, sugars, acids, fruit, fruit juices, or any other component known in the art as a component of, or additive to, a dairy product.

In one aspect of the invention, one or more milk components and/or milk fractions account for at least 50% (weight/weight), such as at least 70%, e.g. at least 80%, preferably at least 90%, of the dairy product.

In one aspect of the invention, one or more milk-based substrates having been treated with an enzyme as defined herein account for at least 50% (weight/weight), such as at least 70%, e.g. at least 80%, preferably at least 90%, of the dairy product.

In one aspect of the invention, the enzyme-treated milk-based substrate is not dried before being used as an ingredient in the dairy product.

In one aspect of the invention, the dairy product is ice cream. In the present context, ice cream may be any kind of ice cream such as full fat ice cream, low fat ice cream, or ice cream based on yoghurt or other fermented milk products. Ice cream may be manufactured by any method known in the art.

In one aspect of the invention, the dairy product is milk or condensed milk.

In one aspect of the invention, the dairy product is UHT milk. UHT milk in the context of the present invention is milk which has been subjected to a sterilization procedure which is intended to kill all microorganisms, including the bacterial spores. UHT (ultra high temperature) treatment may be, e.g., heat treatment for 30 seconds at 130° C., or heat treatment for one second at 145° C.

In one aspect of the invention, the dairy product is ESL milk. ESL milk in the present context is milk which has an extended shelf life due to microfiltration and/or heat treatment and which is able to stay fresh for at least 15 days, preferably for at least 20 days, on the store shelf at 2-5° C.

In another preferred aspect of the invention, the dairy product is a fermented dairy product, e.g., yoghurt.

The microorganisms used for most fermented milk products are selected from the group of bacteria generally referred to as lactic acid bacteria. As used herein, the term “lactic acid bacterium” designates a gram-positive, microaerophilic or anaerobic bacterium, which ferments sugars with the production of acids including lactic acid as the predominantly produced acid, acetic acid and propionic acid. The industrially most useful lactic acid bacteria are found within the order “Lactobacillales” which includes Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp. Additionally, lactic acid producing bacteria belonging to the group of anaerobic bacteria, bifidobacteria, i.e. Bifdobacterium spp. which are frequently used as food cultures alone or in combination with lactic acid bacteria, are generally included in the group of lactic acid bacteria. Lactic acid bacteria are normally supplied to the dairy industry either as frozen or freeze-dried cultures for bulk starter propagation or as so-called “Direct Vat Set” (DVS) cultures, intended for direct inoculation into a fermentation vessel or vat for the production of a fermented dairy product. Such cultures are in general referred to as “starter cultures” or “starters”.

Commonly used starter culture strains of lactic acid bacteria are generally divided into mesophilic organisms having optimum growth temperatures at about 30° C. and thermophilic organisms having optimum growth temperatures in the range of about 40 to about 45° C. Typical organisms belonging to the mesophilic group include Lactococcus lactis, Lactococcus lactis subsp. cremoris, Leuconostoc mesenteroides subsp. cremoris, Pseudoleuconostoc mesenteroides subsp. cremoris, Pediococcus pentosaceus, Lactococcus lactis subsp. lactis biovar, diacetylactis, Lactobacillus casei subsp. casei and Lactobacillus paracasei subsp. paracasei. Thermophilic lactic acid bacterial species include as examples Streptococcus thermophilus, Enterococcus faecium, Lactobacillus delbrueckii subsp. lactis, Lactobacillus helveticus, Lactobacillus delbrueckii subsp. bulgaricus and Lactobacillus acidophilus.

Also the anaerobic bacteria belonging to the genus Bifidobacterium including Bifdobacterium bifidum, Bifdobacterium animalis and Bifdobacterium longum are commonly used as dairy starter cultures and are generally included in the group of lactic acid bacteria. Additionally, species of Propionibacteria are used as dairy starter cultures, in particular in the manufacture of cheese. Additionally, organisms belonging to the Brevibacterium genus are commonly used as food starter cultures.

Another group of microbial starter cultures are fungal cultures, including yeast cultures and cultures of filamentous fungi, which are particularly used in the manufacture of certain types of cheese and beverage. Examples of fungi include Penicillium roqueforti, Penicillium candidum, Geotrichum candidum, Torula kefir, Saccharomyces kefir and Saccharomyces cerevisiae.

In one aspect of the present invention, the microorganism used for fermentation of the milk-based substrate is Lactobacillus casei or a mixture of Streptococcus thermophilus and Lactobacillus delbrueckii subsp. bulgaricus.

Fermentation processes to be used in a method of the present invention are well known and the person of skill in the art will know how to select suitable process conditions, such as temperature, oxygen, amount and characteristics of microorganism/s, additives such as e.g. carbohydrates, flavours, minerals, enzymes, and process time. Obviously, fermentation conditions are selected so as to support the achievement of the present invention.

As a result of fermentation, pH of the milk-based substrate will be lowered. The pH of a fermented dairy product of the invention may be, e.g., in the range 3.5-6, such as in the range 3.5-5, preferably in the range 3.8-4.8.

In one aspect, the use of a herein disclosed cell for producing a product selected from the group consisting of yoghurt, cheese, fermented milk product, dietary supplement and probiotic comestible product, is provided.

In one aspect, the enzymes described herein may be used to prepare cheese products and in methods for making the cheese products. Cheese products may e.g. be selected from the group consisting of cream cheese, cottage cheese, and process cheese.

The treatment of milk products with enzymes that converts lactose into monosaccharides has several advantages. First the products can be consumed by people with lactose intolerance that would otherwise exhibit symptoms such as flatulence and diarreha. Secondly, dairy products treated with lactase will have a higher sweetness than similar untreated products due to the higher perceived sweetness of glucose and galactose compared to lactose. This effect is particularly interesting for applications such as yoghurt and ice-cream where high sweetness of the end product is desired and this allows for a net reduction of carbohydrates in the consumed product. Thirdly, in ice-cream production a phenomenon termed sandiness is often seen, where the lactose molecules crystallizes due to the relative low solubility of the lactose. When lactose is converted into monosaccharides the mouth feeling of the ice-cream is much improved over the non-treated products. The presence of a sandy feeling due to lactose crystallization can be eliminated and the raw material costs can be decreased by replacement of skimmed milk powder by whey powder. The main effects of the enzymatic treatment were increased sweetness.

In one aspect, the enzymes as disclosed herein may be used together with other enzymes such as proteases such as chymosin or rennin, lipases such as phospholipases, amylases, transferases, and lactases. In one aspect, the enzyme as disclosed herein may be used together with a transgalactosylase. This may especially be useful when there is a desire to reduce residual lactose after treatment with the transgalactosylating polypeptide(s) especially at low lactose levels. An enzyme having lactase activity is any glycoside hydrolase having the ability to hydrolyse the disaccharide lactose into constituent galactose and glucose monomers. The group of lactases comprises but is not limited to enzymes assigned to subclass EC 3.2.1.108. Enzymes assigned to other subclasses, such as, e.g., EC 3.2.1.23, may also be lactases. A lactase may have other activities than the lactose hydrolysing activity, such as for example a transgalactosylating activity. In the context of the invention, the lactose hydrolysing activity of the lactase may be referred to as its lactase activity or its beta-galactosidase activity. Enzymes having lactase activity may be of animal, of plant or of microbial origin. Enzymes may be obtained from microbial sources, in particular from a filamentous fungus or yeast, or from a bacterium. The enzyme may, e.g., be derived from a strain of Agaricus, e.g. A. bisporus; Ascovaginospora; Aspergillus, e.g. A. niger, A. awamori, A. foetidus, A. japonicus, A. oryzae; Candida; Chaetomium; Chaetotomastia; Dictyostelium, e.g. D. discoideum; Kluveromyces, e.g. K. fragilis, K. lactis; Mucor, e.g. M. javanicus, M. mucedo, M. subtilissimus; Neurospora, e.g. N. crassa; Rhizomucor, e.g. R. pusillus; Rhizopus, e.g. R. arrhizus, R. japonicus, R. stolonifer; Sclerotinia, e.g. S. libertiana; Torula; Torulopsis; Trichophyton, e.g. T. rubrun; Whetzelinia, e.g. W. sclerotiorum; Bacillus, e.g. B. coagulans, B. circulans, B. megaterium, B. novalis, B. subtilis, B. pumilus, B. stearothermophilus, B. thuringiensis; Bifidobacterium, e.g. B. longum, B. bifidum, B. animalis; Chryseobacterium; Citrobacter, e.g. C. freundii; Clostridium, e.g. C. perfringens; Diplodia, e.g. D. gossypina; Enterobacter, e.g. E. aerogenes, E. cloacae Edwardsiella, E. tarda; Erwinia, e.g. E. herbicola; Escherichia, e.g. E. coli; Klebsiella, e.g. K. pneumoniae; Miriococcum; Myrothesium; Mucor; Neurospora, e.g. N. crassa; Proteus, e.g. P. vulgaris; Providencia, e.g. P. stuartii; Pycnoporus, e.g. Pycnoporus cinnabarinus, Pycnoporus sanguineus; Ruminococcus, e.g. R. torques; Salmonella, e.g. S. typhimurium; Serratia, e.g. S. liquefasciens, S. marcescens; Shigella, e.g. S. flexneri; Streptomyces, e.g. S. antibioticus, S. castaneoglobisporus, S. violeceoruber; Trametes; Trichoderma, e.g. T. reesei, T. viride; Yersinia, e.g. Y. enterocolitica. In one aspect, the lactase is an intracellular component of microorganisms like Kluyveromyces and Bacillus. Kluyveromyces, especially K. fragilis and K. lactis, and other fungi such as those of the genera Candida, Torula and Torulopsis, are a common source of fungal lactases, whereas B. coagulans and B. circulans are well known sources for bacterial lactases. Several commercial lactase preparations derived from these organisms are available such as Lactozym® (available from Novozymes, Denmark), HA-Lactase (available from Chr. Hansen, Denmark) and Maxilact® (available from DSM, the Netherlands), all from K. lactis. All these lactases are so called neutral lactases having a pH optimum between pH 6 and pH 8.

In one aspect of the present invention, a method for production of a lactose free dairy product from a milk-based substrate is presented having the steps of: a.) providing a milk-based substrate; b.) adding an enzyme having neutral lactase activity to the milk-based substrate; c.) pasteurizing the milk-based substrate wherein the lactase having neutral lactase activity retains a substantial amount of activity after said pasteurizing step; and d.) storing the milk-base substrate for a sufficient time to provide a lactose free dairy product.

Preferably, the milk-based substrate has at least 4.7% (w/w) lactose. Preferably, the pasteurizing step is carried out at 65 to 75° C. More preferably, the pasteurizing step is carried out at 70 to 73° C. Most preferably, the pasteurizing step is carried out at 72.8° C.

Preferably, the step of storing is for 3 to 10 days. More preferably, the step of storing is 6 to 9 days. Most preferably, the step of storing is 8 days.

Preferably, the enzyme having neutral lactase activity is derived from a Lactobacillus. More preferably, the enzyme having neutral lactase activity is derived from Lactobacillus delbrueckii bulgaricus. Still more preferably, the has at least about 60% identity to SEQ ID NO:1. Yet more preferably, the enzyme has at least about 60, 65, 70, 75, 80, 85, 90, 91, 92, 93, 94, 95, 96, 97, 98, 99 or 100% identity to SEQ ID NO:1. In still more preferred embodiments, the enzyme is that of SEQ ID NO:1 or a lactase active fragment thereof. In other preferred embodiments, the enzyme having neutral lactase activity is purified. Still more preferably, the enzyme having neutral lactase activity is concentrated. Yet more preferably, the enzyme having neutral lactase activity is in the form of an enzyme preparation which has a reduced level of lipase side activity. In the most preferred embodiments, the enzyme having neutral lactase activity is in the form of an enzyme preparation which has a reduced level of protease, amylase, mannanase, pectinase, cellulase and/or p-nitrobenzylesterase side activities.

### EXAMPLES

The following examples are merely illustrative, and not limiting to this specification in any way.

### Example 1: Enzymes

Experimental Dupont lactase: A thermostable β-galactosidase from Lactobacillus delbrueckii bulgaricus having the amino acid sequence shown in SEQ ID NO:1. As an example of a hydrolyzing lactase from Kluyveromyces lactis, GODO-YNL2 (available from DuPont, Denmark) was used. Another example of a hydrolyzing lactase from Bifidobacterium bifidum is commercially available as Saphera (available from Novozymes, Denmark) or as NOLA Fit (available from Chr. Hansen, Denmark). As an example of a transgalactosylating lactase FoodPro GOS, a Bifidumbacterium bifidum lactase (available from DuPont, Denmark) and the Nutribio GOS L an acid lactase from Aspergillus oryzae (available from DuPont, Denmark) were utilized.

### Example 2: HPLC Lactose Determination

The following method describes the procedure for lactose (and potentially allolactose) quantification in milk samples and other matrices. The samples are derivatized and analysed by HPLC with UV and FLD detection.

Chemicals: Dimethyl sulfoxide, DMSO (CAS: 67-68-5 Sigma A8418); Phosphoric acid, H3PO4, (CAS: 7664-38-2, Sigma P5811); Acetic acid, ≥99% (CAS: 64-19-7, Sigma A6283); Sodium phosphate, NaH₂PO₄, ≥99.0% (CAS: 7558-79-4, Sigma S7907); 4-Amino-benzoic acid, ≥99% (CAS: 150-13-0, Sigma A9878); 2-Methylpyridine borane complex solution, 95% (CAS: 3999-38-0, Sigma 654213); Tetrabutylammonium bisulfate, ≥99.0%, (CAS: 32503-27-8, Sigma 86868); Lactose (CAS: 10039-26-6, Sigma)

The derivatization solvent was DMSO/acetic acid (70/30% v/v) and derivatization reagent 4-Aminobenzoic acid and 2-methylpyridine borane complex in solvent was prepared fresh. Sample solvent was 10 mM Na₂HPO₄ (adjusted to pH 2.5 with H₃PO₄ (85%)).

An Agilent 1100 modular HPLC system equipped with online vacuum degasser, pump, autosampler, column temperature control, diode array detector and fluorescence detector was utilized. Agilent Technologies OpenLAB CDS software was used for quantification. Column: Knauer Prontosil RP-C18 SH (150×4.6 mm, 3 μm) from Mikrolab (KN 15EF180PSG/15VH185PSJ). Tetrabutylammonium hydrogen sulphate was used as the ion-pair reagent in the eluent system. Injection volume was 20 μL, column temperature 20° C., Isocratic: MP A Flow: 0.8 mL/min, A: 10 mM sodium phosphate buffer containing 20 mM tetrabutylammonium bisulfate (pH 2.0) B % acetonitrile for wash program, In between each injection column was washed with 50/50 v/v % acetonitrile/water, Pressure: 250 bar, Runtime: 100 min., Fluorescence Ex: 313 nm and Em: 358 nm, Diode Array Detector absorbance 303 nm. Lactose used for calibration standards was made ranging from 5-500 mg/L in ddH2O.

Sample preparation: L-Arabinose (75 mg/L) prepared in 10 mM NaH₂PO₄ at pH 2.5 was utilized as internal standard for all samples. For lactose free milk and cream and standards: 200 μL sample/standard was transferred to a 1.5 mL Eppendorf tube, added 400 μL sample solvent and mixed. This mixture was centrifuged for 10 min. at max speed (Labnet centrifuge) and 200 μL supernatant was transferred to a 2 mL Eppendorf tube. For Lactose free yoghurt, Skyr and Quarg the following prep. was utilized: Weigh out approximately 1 g of fermented sample to a 1.5 mL Eppendorf tube. Sample was centrifuged for 10 min. at max speed (Labnet centrifuge). 300 μL sample was transferred to a 1.5 mL Eppendorf tube and added 600 μL sample solvent and mixed. This solution was centrifuged for 10 min. at max speed and 200 μL supernatant was transferred to a 1.5 mL Eppendorf tube. Following the samples were derivatized: To the 2 mL eppendorf tube containing 200 μL sample 200 μL was added derivatization reagent. The tube was placed in a Thermomixer at 60° C. to react for 30 min. at 750 rpm. The tube with reaction mixture was removed from the thermomixer after 30 min. and 25 μL reaction mixture was mixed with 225 μL mL mobile phase in a microtiter plate. This solution was filtered through a microtiter plate with filter by centrifuging. Plate was finally sealed before HPLC analysis. Lactose concentration in dairy samples (including milk) was calculated according to the calibration standards and internal control.

### Example 3: Neutral Lactase Activity Determination

The following method is to be used to determine the activity of Lactase activity in NLU/g. Applicable to determination of lactase activity of 2000-5000 neutral lactase units (NLU)/g in enzyme preparations derived from Kluveromyces lactis and Saccharomyces sp. The principle of this assay method is that lactase hydrolyzes o-nitrophenyl-β-D-galactopyranoside (ONPG) in o-nitophenol (ONP) and galactose at 30° C. and pH 6.5. The reaction is stopped with addition of sodium carbonate and the liberated ONP is measured in spectrophotometer or colorimeter at 420 nm. One NLU/g is defined as that quantity of enzyme that liberates 1.30 μM o-nitrophenol/min under assay conditions.

The following reagents was prepared: 0.1 M Magnesium solution. by quantitatively transfer of 24.65 g MgSO4.7H2O (Sigma-aldrich) into 1 L volumetric flask. 5 mM EDTA solution by quantitatively transfer of 1.86 g Na₂EDTA (C₁₀H₁₄N₂Na₂O₈. 2×H₂O) into 1 L volumetric flask, dissolve in small volume of distilled water, and afterwards dilute to volume. 0.1 M PEM buffer: Phosphate buffer solution.—pH 6.5. Quantitatively transfer 8.8 g KH₂PO₄ and 8 g K₂HPO₄.3H₂O into 1 L volumetric flask and dissolve in ca. 900 mL H2O. Add 10 mL Mg solution and 10 mL EDTA solution. Dilute with distilled water and mix. If necessary, adjust pH with aqueous KOH solution 56 g/L or 10% H₃PO₄ solution before use. Na₂CO₃ Stop Solution—Dissolve 50 g of Na₂CO₃ and 37.2 g Na₂EDTA in 1000 ml of distilled water. ONPG Substrate—Dissolve 250 mg of ONPG (Mw: 301.55 g/mol, Sigma-aldrich #N1127) in volumetric flask with buffer to 100 ml. ONP standard solutions—0.02, 0.04, 0.06, 0.08, 0.10, 0.12 and 0.14 μM ONP/mL, prepared as follows; Transfer 139 mg ONP (2-o-nitrophenol, Sigma-aldrich #33444-25G>99% purity, LC grade) into 1 L volumetric flask, and dissolve in 10 mL 95% ethanol. Dilute to volume with H₂O and mix. From this solution pipet 2, 4, 6, 8, 10, 12 and 14 mL aliquots into separate 100 mL volumetric flasks. To each flask add 25 mL Na₂CO₃ solution and dilute to volume with buffer.

Assay Procedure: Weigh in duplicate minimum 1 g lactase enzyme; dissolve in buffer so that in final dilution 1 mL contains 0.027-0.095 NLU. Record total dilution volume. Let the ONPG substrate equilibrate (at least) 10 min at 30° C. Pipet two 1 mL aliquots from each final dilution (sample and sample blank) into separate 15×150 mm test glass tubes. Place test tubes and buffer blank (NOT sample blank) in water bath at 30° C. and equilibrate exactly 5 min. starting at zero time and at 1 min (or 15 sec.) intervals add 5 mL equilibrated ONPG substrate (and mix by shaking, start stop watch when adding the substrate). After 10 min incubation stop the reaction in the same order by 1 min (or 15 sec.) intervals by adding 2 mL Na₂CO₃ solution (or 1 mL if the volumes are scaled down) and mix. Sample blank: Add 2 mL Na₂CO₃ solution (or 1 mL if the volumes are scaled down) to sample blank. Mix by shaking and add 5 mL ONPG substrate. Within 30 min determine OD420 of sample, sample blank, buffer blank and ONP standards with distilled water as blank, e.g. in cuvettes. For each standard solution plot OD420 against μM of ONP. A straight line through the origin must be obtained. Divide absorbance of each standard solution by μM of ONP to obtain absorptivity, a, for each concentration and the average calculated values. A value of 4.65+/−0.05 should be obtained. If this value is not obtained, repeat the analysis with freshly prepared reagents.

Calculate the neutral lactase activity, in the test sample as follows:

$$NLU \text{/} g = \frac{( A \times 8 \times D ) \text{/} ( a \times 10 \times W )}{1.30}$$

A=absorbance of test, corrected for test blank;
8=volume of incubation mixture after termination, mL;
D=total dilution factor of test sample;
a=absorptivity;
10=incubation time, min;
W=weight of test portion, g
1.30=factor used in NLU definition.

Calculate activity as an average of duplicate analysis.

### Example 4: Protein Determination Methods

Protein Determination by Stain Free Imager Criterion

Protein was quantified by SDS-PAGE gel and densitometry using Gel Doc™ EZ imaging system. Reagents used in the assay: Concentrated (2×) Laemmli Sample Buffer (Bio-Rad, Catalogue #161-0737); 26-well XT 4-12% Bis-Tris Gel (Bio-Rad, Catalogue #345-0125); protein markers “Precision Plus Protein Standards” (Bio-Rad, Catalogue #161-0363); protein standard BSA (Thermo Scientific, Catalogue #23208) and SimplyBlue Safestain (Invitrogen, Catalogue #LC 6060. The assay was carried out as follow: In a 96well-PCR plate 50 μL diluted enzyme sample were mixed with 50 μL sample buffer containing 2.7 mg DTT. The plate was sealed by Microseal ‘B’ Film from Bio-Rad and was placed into PCR machine to be heated to 70° C. for 10 minutes. After that the chamber was filled by running buffer, gel cassette was set. Then 10 μL of each sample and standard (0.125-1.00 mg/mL BSA) was loaded on the gel and 5 μL of the markers were loaded. After that the electrophoresis was run at 200 V for 45 min. Following electrophoresis, the gel was rinsed 3 times 5 min in water, then stained in Safestain overnight and finally destained in water. Then the gel was transferred to Imager. Image Lab software was used for calculation of intensity of each band. A calibration curve was made using BSA (Thermo Scientific, Catalogue #23208). The amount of the target protein was determined by the band intensity and calibration curve. The protein quantification method was employed to prepare enzyme samples of used in subsequent Examples.

### Example 5: Generation of Lactase Expressing Bacillus Host Cells Genetically Modified to be Deficient of the Secreted Lipase, lipA

The Bacillus subtilis lipA gene encoding the secreted lipase was deleted in the Bacillus host cells free of p-nitrobenzylesterase, cellulase, pectinase, amylase, protease and mannanase activities [as described in WO2018/187524] using the CRISPR technology. The plasmid pRF827 containing a Cas9 Y155H variant expression cassette was constructed as described in WO/U.S. Ser. No. 18/026,170.

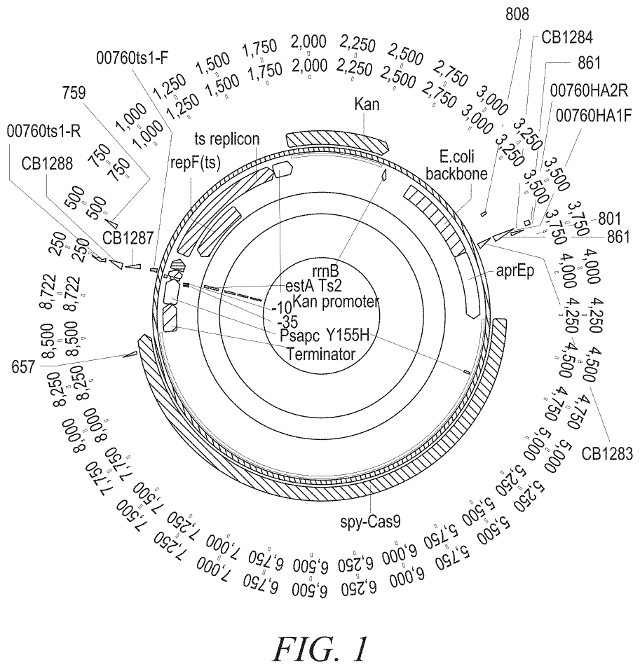

Construction of the pCAS-lipA plasmid (FIG. 1): The plasmid, pRF827 was amplified to replace the serA target site in pRF827 with the lipA target site. The primers, CB1287 (SEQ ID NO:3) and CB1284 (SEQ ID NO:4) were used to amplify the new lipA target site.

CB 1287
(SEQ ID NO: 3)
5′-TTA TGG TTC ACG GTA TTG GAG TTT
TAG AGC TAG AAA TAG CAA GTT-3′
CB 1284
(SEQ ID NO: 4)
5′-GAT AGC AGA AGA AAA TGG AGG
AAT TGT CAG ACC AAG TTT ACT CA-3′

A second DNA fragment was also made using pRF827 and amplified the aprE promoter to the new lipA target site.

CB 1283
(SEQ ID NO: 5)
5′-TGA GTA AAC TTG GTC TGA CAA TTC
CTC CAT TTT CTT CTG CTA TC-3′
CB 1288
(SEQ ID NO: 6)
5′-TCC AAT ACC GTG AAC CAT AAT CCA
CAC ATT ATG CCA CAC-3′

The two pieces were assembled in vitro and subsequently transformed into E. coli TOP10 competent cells. The plasmid contains the lipA target site (SEQ ID NO:7, 5′-TTATGGTTCACGGTATTGGA-3′) which was then used to delete lipA.

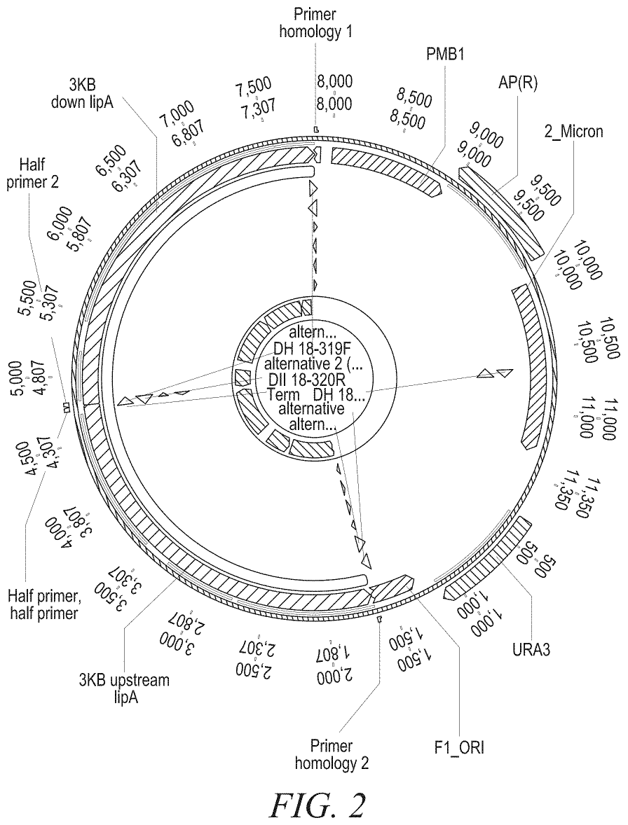

Construction of pRS426-lipA deletion plasmid (FIG. 2): The lipA deletion construct with homology regions in the genome of B. subtilis was assembled using yeast assembly. Four fragments were amplified by PCR and assembled. The first fragment includes half of the pRS426 vector backbone (Genetics. 1989 May; 122(1):19-27). This piece was amplified using primers DH 18-327F and DH 18-273R.

DH 18-327F
(SEQ ID NO: 8)
5′-CTTTTGCAGGACGTGCATTTCAGACTTGTGTAAAGCCTGGGGTGCC
TAATGAGTGAGGTAACTC-3'
DH 18-273R
(SEQ ID NO: 9)
5′-GTAGTAAGAACTATTCATAGAGTGAATCGAAAACAATACG-3′

The second DNA fragment includes half of the pRS426 vector backbone (Genetics. 1989 May; 122(1):19-27). This piece was amplified using primers DH 18-272F and DH 18-325R.

DH 18-272F
(SEQ ID NO: 10)
5′-CGTATTGTTTTCGATTCACTCTATGAATAGTTCTTACTAC-3′
DH 18-325R
(SEQ ID NO: 11)
5′-CTATGGTTCTTCTCAAATGTCACGACTTTCGCTTTCTTCCCTTCC
TTTCTCGCCACGTTCG-3′

The third fragment was amplified using B. subtilis genomic DNA and contains the genetic region upstream of lipA including imrB, imrA, ansZ genes for integration in the genome with overhangs to the region downstream of lipA to allow assembly.

DH 18-317F
(SEQ ID NO: 12)
5′-CGAGAAAGGAAGGGAAGAAAGCGAAAGTCGTGACATTT
GAGAAGAACCATAG-3′
DH 18-320R
(SEQ ID NO: 13)
5′-CTTCAAGGTTTTGTTTTTCATTAATTCTAAGATTCAGA
GGTCATTATTGGTCCT-3′

The fourth fragment was amplified from B. subtilis genomic DNA and contains yczC, yccF, natK genes in the region downstream of lipA with overhangs into the upstream region to allow assembly.

DH 18-316F
(SEQ ID NO: 14)
5′-AGGACCAATAATGACCTCTGAATCTTAGAATTAATGAAAAA
CAAAACCTTGAAGAAT-3′
DH 18-318R
(SEQ ID NO: 15)
5′-TCATTAGGCACCCCAGGCTTTACACAAGTCTGAAATGCACG
TCCTGCAAAAG-3′

These four pieces were assembled and transformed into research yeast strain ATCC #76625=Saccharomyces cerevisiae yph499 using Zymo Research Frozen-EZ Yeast Transformation II Kit. The linear deletion construct which consists of an upstream lipA homology region and a downstream lipA homology region was amplified out of the assembled yeast plasmid using the oligonucleotides DH 18-343F and DH 18-346R. This fragment is used as repair template in deleting lipA.

DH 18-343F
(SEQ ID NO: 16)
5′-CGTGACATTTGAGAAGAACCATAGTACAACGGTG-3′
DH 18-346R
(SEQ ID NO: 17)
5′-GTCTGAAATGCACGTCCTGCAAAAGAAG-3′

Transformation and Isolation of a lipA Deleted Strain

The linear deletion construct and the pCAS-lipA CRISPR plasmid were co-transformed into B. subtilis cells. The transformation reaction was plated on 5 ppm (parts per million) of kanamycin with 1.6% skim milk. Transformants were then streaked on 5 ppm kanamycin plates and a colony PCR was done to check for the lipA deletion (the primers used are below)

DH 18-385F
(SEQ ID NO: 18)
5′-GTCGGTTCGATGAGACCTTCCAC-3′
DH 18-386R
(SEQ ID NO: 19)
5′-CTGCCATGATTCTACGATCTCAC-3′

Transformation of the β-Galactosidase Expression Cassette

The cassette for expression of the 1-galactosidase from Lactobacillus delbrueckii subsp. bulgaricus (SEQ ID NO: 1) was transformed in the lipA deleted strain and plated on X-gal plates. The transformants were reisolated on X-gal plates for a better visual of clones expressing the lactase.

Nucleotide sequence of lipA [B. subtilis 168]
(SEQ ID NO: 20)
ATGAAATTTG TAAAAAGAAG GATCATTGCA CTTGTAACAA
TTTTGATGCT GTCTGTTACA
TCGCTGTTTG CGTTGCAGCC GTCAGCAAAA GCCGCTGAAC
ACAATCCAGT CGTTATGGTT
CACGGTATTG GAGGGGCATC ATTCAATTTT GCGGGAATTA
AGAGCTATCT CGTATCTCAG
GGCTGGTCGC GGGACAAGCT GTATGCAGTT GATTTTTGGG
ACAAGACAGG CACAAATTAT
AACAATGGAC CGGTATTATC ACGATTTGTG CAAAAGGTTT
TAGATGAAAC GGGTGCGAAA
AAAGTGGATA TTGTCGCTCA CAGCATGGGG GGCGCGAACA
CACTTTACTA CATAAAAAAT
CTGGACGGCG GAAATAAAGT TGCAAACGTC GTGACGCTTG
GCGGCGCGAA CCGTTTGACG
ACAGGCAAGG CGCTTCCGGG AACAGATCCA AATCAAAAGA
TTTTATACAC ATC CATTTAC
AGCAGTGCCG ATATGATTGT CATGAATTAC TTATCAAGAT
TAGATGGTGC TAGAAACGTT
CAAATCCATG GCGTTGGACA CATCGGC CTT CTGTACAGCA
GCCAAGTCAA CAGCCTGATT
AAAGAAGGGC TGAACGGCGG GGGCCAGAAT ACGAATTAA
Amino acid sequence of LipA [B.subtilis 168]
(SEQ ID NO: 2)
MKFVKRRIIA LVTILMLSVT SLFALQPSAK AAEHNPVVMV
HGIGGASFNF AGIKSYLVSQGWSRDKLYAV DFWDKTGTNY
NNGPVLSRFV QKVLDETGAK KVDIVAHSMG
GANTLYYIKNLDGGNKVANV VTLGGANRLT TGKALPGTDP
NQKILYTSIY SSADMIVMNY LSRLDGARNVQIHGVGHIGL
LYSSQVNSLI KEGLNGGGQN TN

### Example 6: Expression and Purification of A-Galactosidase from Lactobacillus delbrueckil bulgaricus

Expression of B-galactosidase from Lactobacillus delbrueckii bulgaricus in Bacillus subtilis is achieved under aerobic fermentation conditions. Typically, fermentations are conducted in a cultivation media comprising of carbon and nitrogen sources and inorganic salts at pH 6-8 and at 37 C in bioreactors standard to the industry. β-galactosidase is then recovered from fermentation media using conventional procedures including but not limited to centrifugation, microfiltration, or flocculation combined with rotary vacuum drum or filter press filtration. Supernatant or filtrate is collected and ultrafiltered across 10 kDa PES membranes (Koch) to desired concentration of enrichment for purification. Purification may be employed to reach the desired level of purity such as but not limited to chromatography (i.e. EP2280065), protein crystallization (i.e. WO8908703), or precipitation. Precipitation or crystallization agents for optimal desolubilization include but are not limited to metal halides such as sodium chloride, magnesium chloride, potassium chloride, sodium sulfate and combinations of therein ranging from 0.1%/(w/w) to 5%/(w/w). Chromatography fractions of interest may be pooled and concentrated further to enable a final formulation consisting 40-60% glycerol at pH 6-8. Additionally, precipitated or crystallized β-galactosidase are pelleted at 7000-10,000 rpm where pellets can be resuspended with water and re-pelleted for optimal purity. Pellets can be resuspended with the aforementioned formulation and filtered across a rotary drum filtration or filter press to remove any insoluble particulates formed upstream in the process.

### Example 7: Lactase Temperature Optimum

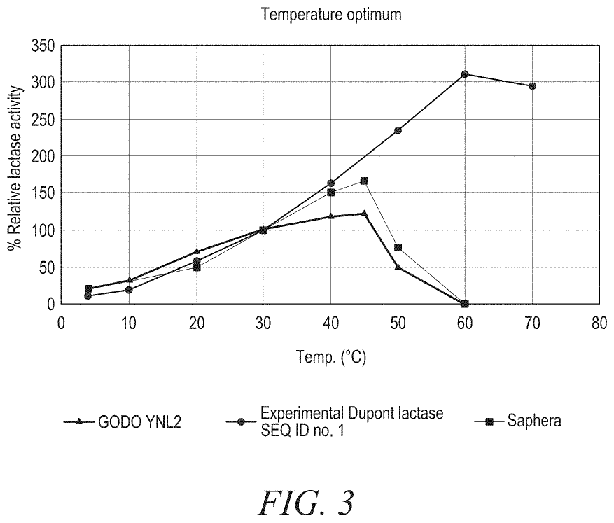

Lactase temperature optimum was assessed from 5° C. to 70° C. for the lactases GODO-YNL, Experimental Dupont lactase SEQ ID NO: 1 and Saphera according to a modified FCC IV method as given in example 3. The results are shown in FIG. 3 and all presented as % relative lactase activity to the enzyme activity quantified at 30° C. Comparing the temperature profile, its clearly seen that the Experimental Dupont lactase SEQ ID NO: 1 has a significant temperature optimum close to 60° C. (300%) whereas the GODO YNL2 and Saphera has an optimum close to 45° C. (100-150%). We expect Experimental Dupont lactase SEQ ID NO: 1 would enable use in milk-based applications at higher temperatures compared to existing hydrolyzing lactases.

### Example 8: Lactase Stability in Milk

Hydrolyzing lactase samples (Experimental Dupont lactase SEQ ID NO: 1 and Nola Fit) were diluted in Aria Mini-milk samples (Arla Foods, Denmark, 0.5% Fat and 4.8% Lactose) and aliquoted to 1 mL samples before incubation at 55° C. and 58° C. for up to 6 hours. One sample was stored at 5° C. for reference. When removed from incubation, samples were quickly cooled on ice and then stored refrigerated until quantification of residual activity.

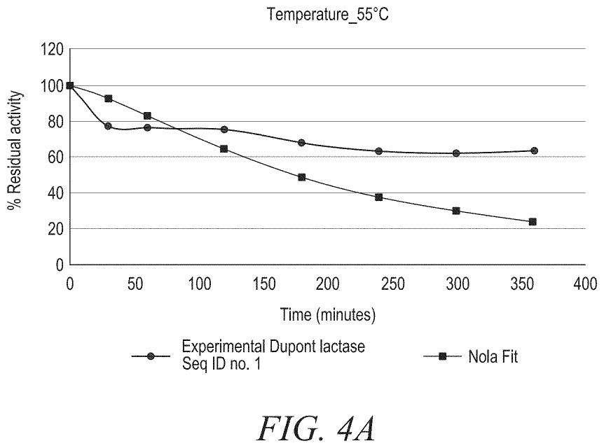

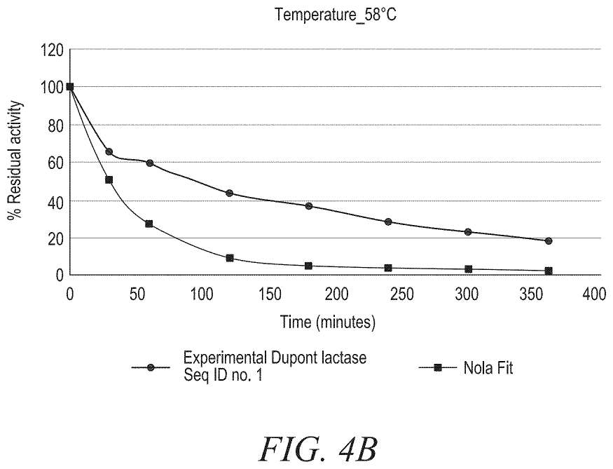

Residual lactase activity was quantified by the following procedure: 20 μL of each milk sample was diluted in 480 μL 0.1 M MES buffer pH 6.4 (Mw: 195.2 g/mol, Sigma-aldrich #M8250-250G). Samples were preheated 2 min to 37° C. before addition of 750 μL 3.7 mg/ml ONPG Substrate (ONPG, Mw: 301.55 g/mol, Sigma-aldrich #N1127). After 10 minutes of enzymation the reaction was stopped with 750 μL 10% Sodium Carbonate (Sigma-aldrich) stop Solution. Samples were further diluted 150 μL in 850 μL stop buffer before reading Abs420 on a spectrophotometer. The % residual activity was calculated as Abs420 (sample incubated at temperature ≥50)/Abs420 (reference sample stored at 5° C.). The results are shown on FIGS. 4A and 4B. It's clearly observed that Experimental Dupont lactase SEQ ID NO: 1 retain significantly higher specific activity compared to Nola Fit both at 55° C. and 58° C., which may enable sufficient lactose hydrolysis in milk-based products.

### Example 9: The Use of a Thermostable Lactase for Production of Lactose Reduced or Free Milk Products at High Temperature

50 mL Arla Mini-milk samples (Arla Foods, Denmark, 0.5% Fat and 4.8% Lactose) was tempered at 60° C. in a water bath. The milk samples were added various lactases to enable lactose reduction at high temperature (60° C.). Protein concentration determined in lactase samples was according method in example 4 and the dosages given in Table 1 were applied.

TABLE 1

| | Protein, | dose |
|---|---|---|
| | mg/ml: | ml/L milk |
| GODO YNL2 | 30.7 | 1.212 |
| NOLA Fit 5500 | 25.2 | 1.476 |
| NutriBio GOS liquid | 21.3 | 1.746 |
| FoodPro GOS | 81.3 | 0.458 |
| Experimental Dupont lactase | 63.0 | 0.590 |
| SEQ ID NO: 1, J15005 | | |

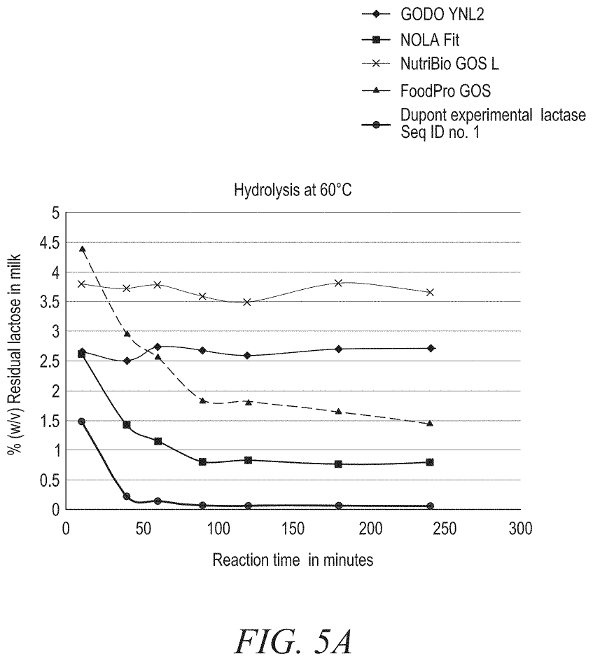

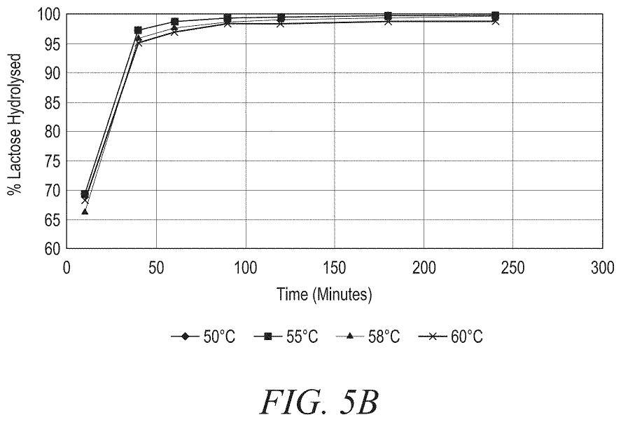

At time zero the various lactase was added milk and mixed. 1 mL sample was extracted at various timepoints ranging from 10 to 240 minutes for following enzyme inactivation. The inactivation was done immediately in a thermomixer for 10 minutes at 98° C. The inactivated samples were stored at −20° C. until quantification. Lactose was quantified in all sample according to the HPLC method in example 2 and the results are shown in FIG. 5A.

It's clear that the high thermostability of Experimental Dupont lactase SEQ ID NO: 1 enabled hydrolysis at 60° C. to efficiently generate lactose free (100 ppm, 0.01% w/vol) or lactose reduced (1000 ppm, 0.1% w/vol) milk samples. The remaining set of lactases tested showed insufficient lactose hydrolysis in milk, likely due to rapid inactivation by the high temperature.

The lactase of SEQ ID NO:1 was also tested using the same conditions but at different temperatures. The results are shown in FIG. 5B. It is clear that the lactase can significantly reduce the amount of lactose at a range of different temperatures.

### Example 10: The Use of a Thermostable Lactase for Lactose Hydrolysis in Condensed Milk

A recombined milk sample with 12% (w/v) lactose was prepared by mixing low fat milk (Arla Foods, Denmark, Fat 0.1%) (83.5%) with skimmed milk powder (Aria Foods, Denmark, Fat 1.25%, Batch 3150035537) (16.5%). The milk sample was tempered at 55° C. before adding below indicated doses in table 2 of the Experimental Dupont lactase SEQ ID NO: 1. After exactly 1 hour the milk was quickly heated to 97° C. for 10 min to inactivate the lactase. Residual lactose was quantified according to the HPLC method in example 2 and the results are shown in table 2. The experiment clearly illustrates the ability of the Experimental Dupont lactase SEQ ID NO: 1 to reduce lactose at high temperature in a milk-based solution.

TABLE 2

| | Residual |
|---|---|
| Enzyme | lactose |
| dosage | in % |
| g/L milk | (w/v) |
| 0.1 | 6.000 |
| 0.2 | 4.200 |
| 0.3 | 2.300 |
| 0.4 | 1.700 |
| 3.4 | 0.078 |
| 3.6 | 0.072 |
| 3.8 | 0.067 |

### Example 11: The Use of a Thermostable Lactase for Production of Recombined Sweet Condensed Milk

Procedure; Sample no. 2 to 8 was produced according to ingredient (in % w/w) table 3. Skimmed milk powder (Arla Foods, Denmark, Fat 1.25%, Batch 3150035537) (22.9% w/w) and demineralized water (24.4% w/w) was mixed at 55° C. under good agitation and hydrated for approximately 15 minutes for complete solubilization. Experimental Dupont lactase: J15005 lot:4863191499 corresponding to Lactase SEQ ID NO: 1 (having an lactase activity of 16976 SDLU/g) was added in various concentration to sample 2 to 6 (0.005, 0.01, 0.34, 0.50 and 0.34% w/w) and the mix was further incubated for 60 min at 55° C. Butter Oil AMF (Butter oil, Corman, Belgium no. A00015111) was melted and then added to the mix together with sucrose (Granulated sugar, 550, Nordic Sugar, Denmark) and Recodan (RECODAN® RS VEG, Dupont Nutrition Bioscience, Denmark) at 75° C. (only 65° C. for trial 6).

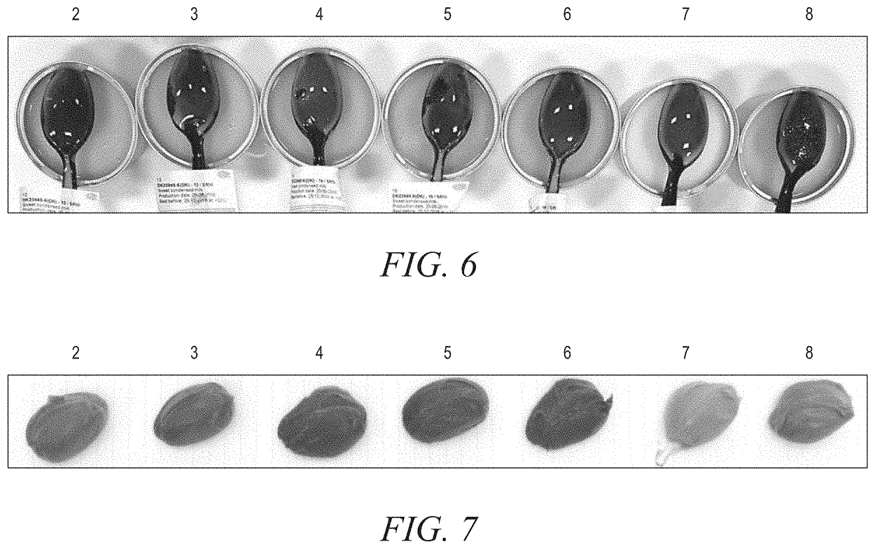

All samples were then homogenized at 75° C. (only 65° C. for sample 6), at 80 bar and then further pasteurized at 90° C. for 15 seconds. The sweet condensed milk was then filled on aluminum cans and sealed. Lactose powder (Variolac® 992 BG100, Arla Foods, Denmark) was grinded as fine as possible ensuring only small crystal formation and added only sample 7 post pasteurization and was incubated for 60-90 min at 32° C. before filling. Lactose was quantified on sample 2-6 according to the HPLC method in example 2 and the results are shown in table 4. The lactose concentration is clearly reduced according to the added amount of thermostable lactase enzyme. A picture of the recombined sweet condensed milk is show in FIG. 6. The picture clearly shows the lactose crystal formation in sample 8 where no Experimental Dupont lactase enzyme SEQ ID NO: 1 nor fine grinded lactose (microcrystalline) has been used (that can be applied to avoid larger crystal formation). Sample 2 and 7 had a slight sandy mouthfeel but sample 3-6 with the Experimental Dupont lactase SEQ ID NO: 1 was all smooth in mouthfeel and without any crystal formation. The data clearly demonstrate that use of the thermostable Experimental Dupont lactase SEQ ID NO: 1 at high temperature may surprisingly prevent lactose crystal formation and reduce sandy mouthfeel for production of recombined sweet condensed milk.

TABLE 3
Ingredients in %

| | | 2 | 3 | 4 | 5 | 6 | 7 | 8 |
|---|---|---|---|---|---|---|---|---|
| Water (Ion) | % | 24.4 | 24.4 | 24.1 | 24.0 | 24.1 | 24.4 | 24.5 |
| Skimmed milk powder | % | 22.9 | 22.9 | 22.9 | 22.9 | 22.9 | 22.9 | 22.9 |
| Sucrose | % | 43.5 | 43.5 | 43.5 | 43.5 | 43.5 | 43.5 | 43.5 |
| Butter oil (AMF) | % | 9.0 | 9.0 | 9.0 | 9.0 | 9.0 | 9.0 | 9.0 |
| (17) RECODAN RS VEG | % | 0.2 | 0.2 | 0.2 | 0.2 | | 0.2 | 0.2 |
| (17) RECODAN ™ RS 305 | % | | | | | 0.2 | | |
| (6) Lactose (Variolac ® 992 BG100) | % | | | | | | 0.1 | |
| Experimental Dupont lactase: J15005 lot: 4863191499 | % | 0.005 | 0.010 | 0.340 | 0.500 | 0.340 | | |
| Total % | | 100.0 | 100.0 | 100.0 | 100.0 | 100.0 | 100.0 | 100.0 |

TABLE 4

| Trial number | 2 | 3 | 4 | 5 | 6 |
|---|---|---|---|---|---|
| % Experimental Dupont | 0.005 | 0.01 | 0.34 | 0.50 | 0.34 |
| lactase Seq ID NO: 1: | | | | | |
| Lactose content in ppm: | 23000 | 1600 | 98 | 85 | 120 |

Cooked sweet condensed milk procedure: Aluminum cans with samples prepared above was heat treated 10 min at 120° C. in an autoclave to induce maillard reaction. (cooked to obtain maillard reaction). Samples were afterwards stored at room temperature for 6 weeks. A picture of the cooked sweet condensed milk is shown in FIG. 7.

The picture clearly shows that same level of wanted browning compared to control without microcrystalline lactose or added lactase can surprisingly be achieved with a dose of only 0.05% (w/w) Experimental Dupont lactase SEQ ID NO: 1 that prevented crystal formation. For all doses above 0.05% of Experimental Dupont lactase (sample 3-6) an increased browning could be observed as a response to increased maillard reaction.

### Example 12: The Use of a Thermostable Lactase for Production of Low Lactose Containing Milkshake

The use of a thermostable lactase for production of low lactose milkshake was tested in following example. All liquid ingredients according to table 5 was mixed at 55° C.: Cream (Arla Foods, Denmark, 38% fat), Skimmed milk (Arla Foods, Denmark, 0.1% Fat, 4.8% Lactose) and lactase enzymes (Experimental Dupont lactase enzyme SEQ ID NO: 1, Nola Fit or no enzyme). The dry ingredients were mixed in: Skimmed milk powder (Aria Foods, Denmark, Fat 1.25%, Batch 3150035537), sucrose (Granulated sugar, 550, Nordic Sugar, Denmark), Litesse® Two (Dupont, Denmark), Cremodan MSA (Dupont, Denmark) and following Vanilla flavouring and colouring (Annatto Seed Extract, Danisco, Denmark, P. no. 1211663) was added. All ingredients were thoroughly mixed and hold at 56° C. for 70 minutes. The temperature was increased to 70° C. and the three samples (1, 2 and 3) was homogenized at: 70° C./pressure according to fat percentage and following pasteurized at: 84° C. for 30 sec. Samples was afterwards cooled to 5° C. and filled to 2×180 ml TPS+2×155 ml pots and the rest in a metal container for further heat treatment. Metal containers was heated to 95° C. for 10 min to ensure proper enzyme inactivation. Lactose was quantified according to example 2 and Sample 1, 2 and 3 contained: 4.50% (w/v), 0.17% (w/v) and 0.30% (w/v) lactose, respectively. This clearly demonstrate the efficient use of Experimental Dupont lactase enzyme SEQ ID NO: 1 for low lactose milk-shake at high temperature.

TABLE 5

| Ingredients % | | Sample 1 | Sample 2 | Sample 3 |
|---|---|---|---|---|
| Cream 38% fat | % | 9.093 | 9.093 | 9.093 |
| Skimmed milk | % | 77.02 | 76.829 | 76.829 |
| Skimmed milk powder | % | 3.138 | 3.158 | 3.158 |
| Sucrose | % | 10 | 7.9 | 7.9 |
| Litesse ® Two | % | 0 | 2.1 | 2.1 |
| CREMODAN MSA | % | 0.6 | 0.6 | 0.6 |
| S-Vanilla 507441 T | % | 0.1 | 0.1 | 0.1 |
| Annatto Extract | % | 0.05 | 0.05 | 0.05 |
| Experimental Dupont lactase | % | 0 | 0.17 | 0 |
| enzyme SEQ ID NO: 1 | | | | |
| Nola fit | % | 0 | 0 | 0.17 |
| | | 100.000 | 100.000 | 100.000 |

### Example 13: The Use of a Thermostable Lactase for Production of Low Lactose Ice Cream

The use of a thermostable lactase for production of low lactose icecream was tested in following example. All liquid ingredients according to table 6 was mixed at 55° C.: Cream (Arla Foods, Denmark, 38% fat), Skimmed milk (Arla Foods, Denmark, 0.1% Fat, 4.8% lactose), Glucose syrup (32DE 95% TS), and lactase enzymes (Experimental Dupont lactase enzyme SEQ ID NO: 1, Nola Fit or no enzyme). The dry ingredients were mixed in: Skimmed milk powder (Arla Foods, Denmark, Fat 1.25%, Batch 3150035537), sucrose (Granulated sugar, 550, Nordic Sugar, Denmark), Litesse® Two (Dupont, Denmark), Cremodan MSA (Dupont, Denmark), Cremodan® SE 30 (Dupont, Denmark) and following flavouring (Vanilla flavouring) and colouring (Annatto Seed Extract, Danisco, Denmark, P. no. 1211663) was added. All ingredients were thoroughly mixed and hold at 56° C. for 70 minutes. The temperature was increased to 70° C. and the three samples (1, 2 and 3) was homogenized at: 70° C./pressure according to fat percentage and following pasteurized at: 84° C. for 30 sec. Samples was afterwards cooled to 5° C. and filled to 2×180 ml TPS+2×155 ml pots and the rest in a metal container for further heat treatment. Metal containers was heated to 95° C. for 10 min to ensure proper enzyme inactivation and following frozen. Lactose was quantified according to example 2 and Sample 1, 2 and 3 contained: 4.90% (w/v), 0.20% (w/v) and 0.74% (w/v) lactose, respectively. This clearly demonstrate the efficient use of Experimental Dupont lactase enzyme SEQ ID NO: 1 for low lactose ice-cream at high temperature.

TABLE 6

| Ingredients | | Sample 1 | Sample 2 | Sample 3 |
|---|---|---|---|---|
| Cream 38% fat | % | 20.929 | 20.929 | 20.929 |
| Skimmed milk | % | 57.34 | 57.262 | 57.262 |
| Skimmed milk powder | % | 5.031 | 5.039 | 5.039 |
| Sucrose | % | 12 | 10 | 10 |
| Glu. syr. p. 32DE 95% TS | % | 4 | 0 | 0 |
| Litesse ® Two | % | 0 | 5.9 | 5.9 |
| CREMODAN MSA | % | 0 | 0 | 0 |
| CREMODAN ® SE 30 | % | 0.55 | 0.55 | 0.55 |
| S-Vanilla 507441 T | % | 0.1 | 0.1 | 0.1 |
| Annatto Extract | % | 0.05 | 0.05 | 0.05 |
| Experimental Dupont lactase | % | 0 | 0.17 | 0 |
| enzyme SEQ ID NO: 1 | | | | |
| Nola fit | % | 0 | 0 | 0.17 |
| | | 100.000 | 100.000 | 100.000 |

### Example 14: Sensory Evaluation

Production of UHT Milk for Sensory Evaluation

Add the different enzyme to the milk and leave for 24 hours in ice water bath at 5 degree C. with circulation.

UHT-PHE Direct Injection:
H1: 70° C.
H2: 90° C.
Injection: 142° C. 3 sec
Vacuum: 87° C.
Downstream Homogenization (150/30)
C2: 45° C.
C3: 20° C.
Fill in 1 L sterile bottles
Store at room temperature for 10 weeks

Sensory Method

To describe the impact on sensory perceivable product attributes, descriptive sensory analysis is chosen. The basis for the descriptive analysis is ISO 13299 “Sensory analysis—Methodology—General guidance for establishing a sensory profile”.

In the sensory descriptive analysis, the intensity of each descriptor is evaluated on a line scale with two anchor points indicating low and high intensity, respectively. The anchor points for low and high is taught to the panel in the training/calibration sessions. All samples are evaluated in triplicate.

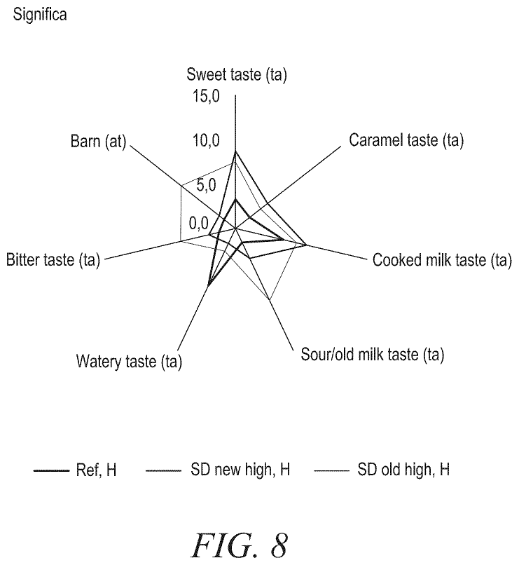

The sensory panel consists of 8 persons, who have all passed the basic sensory screening test before they are accepted in the panel. Before taking part in the descriptive analysis of this analysis. The panelists are trained in recognizing and intensity scaling of the product attributes. FIG. 8 shows flavor attributes with a significant difference. It clearly depicts how a lactase containing LipA will cause barn like, sour, old, and bitter taste in the milk whereas a sample without LipA whether being reference milk or milk added a lactase without LipA does not. Flavor attribute in UHT milk like sweet, cooked and caramel are normally seen with addition of lactase.

Lipase/FFA Testing

### Reagent Preparation

R1: Prepare R1 by mixing one bottle of Color A and Solvent A.

After preparing the R1, store at 2-10° C. and use within 1 month.

R2: Prepare R2 by mixing one bottle of Color B and Solvent B.

After preparing the R2, store at 2-10° C. and use within 1 month.

Procedure

200 μL enzyme samples were added to 800 μL reference milk with 3,5% fat. Samples were then stored at 5° C. for 20 hours. 10 μl of reference milk and enzymated milk samples were added 297 μL R1 and incubation at 37 C in thermomixer. After 10 min 150 μL R2 was added to each tube and incubation at 37° C. continued for 8 minutes. Samples were then centrifuged leaving the milk haze in the top fraction. 200 μL was carefully aliquoted from the bottom fraction before OD 546 was read in microtiter plates.

Results showed OD546 of 0.5673 for lactase containing LipA, 0.2213 for the milk reference, and 0.3048 for lactase without LipA.

### Example 15: Addition of Lactase after Pasteurization and Homogenization

Pre-pasteurised (72° C. for 15 s) bulk blended skimmed milk (0.1% fat) (Arla Foods, Denmark) stored at 4-6° C. is standardized to a desired protein (4% w/w) and fat (1% w/w) content by the addition of skimmed milk powder (33% protein, 1.2% fat, 54% carbohydrate) from BBA Lactalis (Laval, Mayenne, France) and cream (38% fat) from Arla Foods, Denmark. The standardized milk is then pasteurized and homogenized in a standard plate heat exchange pasteurizer. Homogenization is performed at 65° C. at 200 bar and pasteurization at 95° C. for 6 minutes. The milk is then subsequently cooled to its fermentation temperature (45° C.). After reaching the fermentation temperature, the milk is inoculated with a thermophilic starter culture for example, YO-MIX 224/485 at an inoculation rate of 20 DCU/100 L. The lactase is added directly with the culture. Alternatively, the lactase can be added after the pasteurization and homogenization (i.e. during the cooling to the fermentation temperature) at a temperature ≤60° C. After the enzyme addition and during the fermentation, heated (95° C., 20 min) and unheated samples (2 mL each) are taken over various time-points. The unheated samples were used for the determination of the remaining lactase activity. Residual lactase activity was quantified by the following procedure: 20 μL of each milk sample was diluted in 480 μL 0.1 M MES buffer pH 6.4 (Mw: 195.2 g/mol, Sigma-aldrich #M8250-250G). Samples were preheated 2 min to 37° C. before addition of 750 μL 3.7 mg/ml ONPG Substrate (ONPG, Mw: 301.55 g/mol, Sigma-aldrich #N1127). After 10 minutes of enzymation the reaction was stopped with 750 μL 10% Sodium Carbonate (Sigma-aldrich) stop Solution. Samples were further diluted 150 μL in 850 μL stop buffer before reading Abs420 on a spectrophotometer. The % residual activity was calculated as Abs420 (sample incubated at temperature ≥50)/Abs420 (reference sample stored at 5° C.). Heated samples were used for the later determination of the remaining lactose according to Example 2. Fermentation was followed using the CINAC multichannel pH system (Ysebaert, Frdpillon, France), which monitors the pH development every 5 min. Fermentation was conducted until pH 4.60 and the product was cooled on a yogurt plate heat exchanger (SPX Flow Technology, Sussex, UK) and YTRON-ZP shear-pump system (YTRON Process Technology, Bad Endorf, Germany) to 24° C. The resulting stirred style yogurts was stored at 4-6° C.

The words “comprise”, “comprises” and “comprising” are to be interpreted inclusively rather than exclusively. This interpretation is intended to be the same as the interpretation that these words are given under United States patent law at the time of this filing.

The singular forms “a” and “an” are intended to include plural referents unless the context dictates otherwise. Thus, for example, a reference to the presence of “an excipient” does not exclude the presence of multiple excipients unless the context dictates otherwise.

### Example 16: HPLC Method for Quantification of GOS Fiber

It is already described that a Lactobacillus delbrueckii bulgaricus lactase is capable of GOS formation (DP3+) in skimmed milk with 5.2% initial lactose (ref 1, 2) converting 15% of the lactose to GOS with a degree of polymerization of 3 or above (DP3+). Tien-Thanh Nguyen et al. (ref 3) also describe testing a Lactobacillus delbrueckii bulgaricus lactase at higher substrate concentrations converting op to 50% of the lactose into total GOS (TGOS, including DP2 GOS like allolactose) in buffered conditions with 20.5% initial lactose. It has however not been expected that similar conversion yields in milk substrates could be achieved. Todor Vasiljevic and Paul Jelen 2003 (ref 4) showed that the yields using a Lactobacillus delbrueckii bulgaricus lactase was about 40% lower in milk substrate of 5% lactose compared to using 5% lactose dissolved in buffer as substrate. The same trend of lower conversion of lactose into TGOS in milk substrate compared to buffered conditions was observed when testing at 10-15% lactose concentrations.

We show here that the Lactobacillus delbrueckii bulgaricus lactase is commercially relevant for in situ GOS production when enzymating a milk substrate. Surprisingly, we found that it would be possible to obtain more than 25% sugar reduction in a milk based substrate of 9-30% (w/w) lactose. A 25% sugar reduction would not be possible when using a regular lactase like the GODO YNL2.

The standard lactose (HPLC analytical grade, Sigma Aldrich) was prepared in double distilled water (ddH2O) and filtered through 0.2 μm syringe filters. A dilution series ranging from 500 to 10000 ppm of the lactose standard was created.

Similar sample preparations of milk-base solutions were applied, however, the milk sample was diluted to approximately 5% milk carbohydrate, then 200 mg sample (weight noted) in 800 μL H₂O was mixed thoroughly. 50 μL Carrez reagent A (Carrez Clarification Kit, 1.10537.001, Merck) and 50 μL Carrez B was added to 1000 μL diluted sample with mixing after each to induce protein and lipid precipitation. The sample mixture was incubated 15 minutes at room temperature and 50 μL 10 mM NaOH, 1 mM EDTA was added to the sample. The sample was centrifuged at 10.000 rpm for 4 minutes and 300 μL clarified supernatant was transferred to an MTP filter plate, through 0.20 μm 96 well plate filters (centrifuged 3000 rpm in 15 minutes) before analysis (Corning filter plate, PVDF hydrophile membrane, NY, USA). All samples were analyzed in duplicate and in 96 well MTP plates sealed with tape.

Instrumentation:

Quantification of galacto-oligosaccharides (GOS), lactose, glucose and galactose were performed by HPLC. Analysis of samples was carried out on a Dionex Ultimate 3000 HPLC system (Thermo Fisher Scientific) equipped with a DGP-3600SD Dual-Gradient analytical pump, WPS-3000TSL thermostated autosampler, TCC-3000SD thermostated column oven, and a RI-101 refractive index detector (Shodex, JM Science). Chromeleon datasystem software (Version 6.80, DU10A Build 2826, 171948) was used for data acquisition and analysis.

Chromatographic Conditions:

The samples were analyzed by HPLC using an RSO oligosaccharide column, Ag⁺ 4% crosslinked (Phenomenex, The Netherlands) equipped with an analytical guard column (Carbo-Ag⁺ neutral, AJO-4491, Phenomenex, The Netherlands) operated at 70° C. The column was eluted with double distilled water (filtered through a regenerated cellulose membrane of 0.45 μm and purged with helium gas) at a flow rate of 0.3 ml/min.

Isocratic flow of 0.3 ml/min was maintained throughout analysis with a total run time of 45 minutes and injection volume was set to 10 μL. Samples were held at 30° C. in the thermostated autosampler compartment to ensure solubilization of all components. The eluent was monitored by means of a refractive index detector (RI-101, Shodex, JM Science) and quantification was made by the peak area relative to the peak area of lactose standard as described above. Peaks of DP 3 and higher (DP3+) were quantified as galacto-oligosaccharides (DP3, DP4, DP5 and so forth). The assumption that all DP3+ galacto-oligosaccharides components provide same response was confirmed with mass balances. Lactose including other DP2 components was quantified as DP2, glucose and galactose in a similar manner.

### Example 17: Total GOS

Total GOS (TGOS) is the total sum of transgalactosylated molecules. The value is calculated based of amount of galactose bound in TGOS molecules multiplied with a factor of 1.4 in which the factor of 1.4 represents the galactose to glucose ratio. The galactose bound in TGOS molecules is calculated by subtracting free galactose and galactose bound in lactose from total galactose (which is the sum of free galactose, galactose bound in TGOS and galactose bound in lactose). This calculation approach is in line with AOAC 2001.02 method.

Based on values obtained from example 2 and example 16 we calculate the TGOS as follows;

TGOS=(Total carb.(% w/w)/1.9−galactose(% w/w)−Lactose(% w/w)/1.9)×1.4

Total carb. (% w/w) is the sum of DP3+(% w/w), DP2 (% w/w), glucose (% w/w) and galactose (% w/w)

Total carb. (% w/w)/1.9 represents total galactose in the sample

galactose (% w/w) is the free galactose in the sample.

Lactose (% w/w)/1.9 is the galactose bound in the lactose molecule

### Example 18: Lactose Conversion to GOS in Lactose Buffered Substrate

Lactose solutions of 12, 20 and 30% were prepared, of anhydrate Lactose (Sigmaaldrich, #17814) in 100 ml 0.1 M MES buffer pH 6.4 (Mw: 195.2 g/mol, Sigmaaldrich #M8250-250G). The solution temperature was raised to 40° C. for addition of GODO YNL2 or 55° C. for addition of LBul. When the correct temperature was reached the enzymes were added at time 0 according to table 7. After 20, 40, 60 and 80 minutes, 5 ml sample was collected for inactivation. The inactivation was done at 95° C. for 10 minutes. Quantification of DP3+, DP2, glucose, galactose was done according to example 16 and lactose was quantified according to example 2.

TABLE 7

| % Lactose | Enzyme | Dose g/L | Temp. (° C.) |
|---|---|---|---|
| 12 | LBul | 0.77 | 55 |
| 20 | LBul | 1.29 | 55 |
| 30 | LBul | 1.43 | 55 |
| 12 | GODO YNL2 | 0.79 | 40 |
| 20 | GODO YNL2 | 8.00 | 40 |
| 30 | GODO YNL2 | 11.9 | 40 |

All data is presented in table 8 for LBul and table 9 for GODO YNL2 together with calculated values of Total carbohydrates (carb.), % sugar reduction, TGOS (according to example 17) and % lactose converted to TGOS.

TABLE 8
LBul lactose conversion to GOS in Lactose buffered substrate

| | | | | | | | | | TGOS (% w/w) | % lactose |
|---|---|---|---|---|---|---|---|---|---|---|
| Initial | | DP3+ (% | DP2 (% | Glucose (% | Galactose (% | Total carb. (% | lactose | % sugar | (AOAC | converted |
| lactose | Sample | w/w) | w/w) | w/w) | w/w) | w/w) * | (% w/v) | reduction** | 2001.02 calc) | to TGOS*** |
| ~12% | 55° C., | 4.48 | 5.57 | 2.34 | 1.14 | 13.52 | 4.50 | 33.11 | 5.05 | 37.38 |
| lactose | 20 min | | | | | | | | | |
| | 55° C., | 4.98 | 4.08 | 3.05 | 1.54 | 13.65 | 2.00 | 36.48 | 6.42 | 47.06 |
| | 40 min | | | | | | | | | |
| | 55° C., | 4.99 | 3.36 | 3.40 | 1.80 | 13.55 | 0.94 | 36.80 | 6.77 | 49.95 |
| | 60 min | | | | | | | | | |
| | 55° C., | 4.79 | 3.03 | 3.65 | 2.01 | 13.48 | 0.52 | 35.55 | 6.74 | 49.99 |
| | 80 min | | | | | | | | | |
| ~20% | 55° C., | 6.88 | 9.22 | 3.69 | 1.46 | 21.25 | 7.10 | 32.35 | 8.38 | 39.44 |
| lactose | 20 min | | | | | | | | | |
| | 55° C., | 7.88 | 6.95 | 4.81 | 2.02 | 21.66 | 3.30 | 36.37 | 10.70 | 49.40 |
| | 40 min | | | | | | | | | |
| | 55° C., | 7.88 | 5.76 | 5.32 | 2.36 | 21.31 | 1.70 | 36.96 | 11.15 | 52.33 |
| | 60 min | | | | | | | | | |
| | 55° C., | 7.78 | 5.33 | 5.75 | 2.73 | 21.58 | 0.90 | 36.04 | 11.42 | 52.93 |
| | 80 min | | | | | | | | | |
| ~30% | 55° C., | 10.01 | 14.27 | 5.30 | 1.82 | 31.40 | 11.00 | 31.88 | 12.49 | 39.77 |
| lactose | 20 min | | | | | | | | | |
| | 55° C., | 11.57 | 10.73 | 6.87 | 2.52 | 31.69 | 5.10 | 36.50 | 16.07 | 50.70 |
| | 40 min | | | | | | | | | |
| | 55° C., | 11.86 | 9.10 | 7.77 | 3.05 | 31.78 | 2.70 | 37.32 | 17.15 | 53.99 |
| | 60 min | | | | | | | | | |
| | 55° C., | 11.77 | 8.43 | 8.43 | 3.53 | 32.15 | 1.90 | 36.61 | 17.36 | 53.98 |
| | 80 min | | | | | | | | | |

* Total carb. (% w/w) is the sum of DP3+ (% w/w), DP2 (% w/w), glucose (% w/w) and galactose (% w/w)
**% lactose converted to TGOS is calculated as % TGOS relative to total carb.
***% sugar reduction is % DP3+ relative to Total carb.

TABLE 9
GODO YNL2 lactose conversion to GOS in Lactose buffered substrate

| | | | | | | | | | TGOS (% w/w) | % lactose |
|---|---|---|---|---|---|---|---|---|---|---|
| Initial | | DP3+ (% | DP2 (% | Glucose (% | Galactose (% | Total carb. (% | lactose (% | % sugar- | (AOAC | converted |
| lactose | Sample | w/w) | w/w) | w/w) | w/w) | w/w) * | w/v) | reduction ** | 2001.02 calc) | to TGOS *** |
| ~12% | 40° C., | 0.89 | 12.51 | 0.55 | 0.36 | 14.31 | 12.00 | 6.23 | 1.20 | 8.36 |
| lactose | 20 min | | | | | | | | | |
| | 40° C., | 1.07 | 12.35 | 0.57 | 0.36 | 14.34 | 12.00 | 7.48 | 1.22 | 8.54 |
| | 40 min | | | | | | | | | |
| | 40° C., | 1.03 | 12.28 | 0.58 | 0.42 | 14.31 | 12.00 | 7.21 | 1.11 | 7.76 |
| | 60 min | | | | | | | | | |
| | 40° C., | 0.99 | 12.19 | 0.62 | 0.41 | 14.22 | 12.00 | 6.99 | 1.05 | 7.42 |
| | 80 min | | | | | | | | | |
| ~20% | 40° C., | 4.16 | 11.84 | 4.62 | 2.16 | 22.63 | 8.50 | 18.38 | 7.39 | 32.67 |
| lactose | 20 min | | | | | | | | | |
| | 40° C., | 4.78 | 8.32 | 6.21 | 3.05 | 22.36 | 3.80 | 21.37 | 9.40 | 42.06 |
| | 40 min | | | | | | | | | |
| | 40° C., | 4.62 | 7.10 | 7.23 | 3.81 | 22.76 | 2.00 | 20.31 | 9.96 | 43.76 |
| | 60 min | | | | | | | | | |
| | 40° C., | 4.06 | 6.29 | 7.73 | 4.41 | 22.49 | 1.00 | 18.06 | 9.66 | 42.96 |
| | 80 min | | | | | | | | | |
| ~30% | 40° C., | 7.22 | 15.35 | 7.62 | 3.09 | 33.49 | 10.00 | 21.56 | 12.98 | 38.77 |
| lactose | 20 min | | | | | | | | | |
| | 40° C., | 7.80 | 11.07 | 9.89 | 4.51 | 33.28 | 4.10 | 23.44 | 15.18 | 45.62 |
| | 40 min | | | | | | | | | |
| | 40° C., | 7.11 | 9.42 | 10.88 | 5.49 | 32.91 | 1.80 | 21.62 | 15.23 | 46.30 |
| | 60 min | | | | | | | | | |
| | 40° C., | 6.17 | 8.65 | 11.50 | 6.37 | 32.69 | 1.10 | 18.87 | 14.36 | 43.92 |
| | 80 min | | | | | | | | | |

* Total carb. (% w/w) is the sum of DP3+ (% w/w), DP2 (% w/w), glucose (% w/w) and galactose (% w/w)
** % lactose converted to TGOS is calculated as % TGOS relative to total carb.
*** % sugar reduction is % DP3+ relative to Total carb.

Conclusion:

It is clear, that unlike the GODO YNL2 lactase used to produce lactose free products, LBul is capable of producing significant levels of GOS fibers (DP3+) thereby providing above 30% sugar reduction in lactose buffered substrates of 12-30%.

### Example 19: Lactose Conversion to GOS in Milk Based Substrate

Substrates of either Arla Mini-milk (Arla Foods, Denmark, 0.5% Fat and 4.8% Lactose) or skimmed milk powder (Aria Foods, Denmark, Fat 1.25%, Batch 3150035537), according to table 10, were prepared in water (100 ml) and the temperature maintained at 50° C. for the Mini-milk, at 55° C. for the 12-40% milk powder and at 58° C. for the 60% milk powder. The LBul was dosed at time 0 according to table 10 and at various timepoints a sample was collected for heat inactivation. Inactivation was done for 10 minutes at 95° C. Quantification of DP3+, DP2, glucose, galactose was done according to example 16 and lactose was quantified according to example 2.

TABLE 10

| | | Enzyme |
|---|---|---|
| % lactose | Substrate | Dose (g/L) |
| 4.8% | Aria Mini-milk | 0.30 |
| 6% lactose | 12% skimmed milk powder | 0.39 |
| 9% lactose | 18% skimmed milk powder | 0.58 |
| 12% lactose | 24% skimmed milk powder | 0.77 |
| 20% lactose | 40% skimmed milk powder | 1.29 |
| 30% lactose | 60% skimmed milk powder | 1.43 |

All data is presented in table 11 together with calculated values of Total carbohydrates (carb.), % sugar reduction, TGOS (according to example 17) and % lactose converted to TGOS.

TABLE 11
LBul lactose conversion to GOS in milk substrate

| Initial | | | | | | | | | | |
|---|---|---|---|---|---|---|---|---|---|---|
| lactose | | | | | | | | | TGOS (% w/w) | % lactose *** |
| concen | | DP3+ (% | DP2 (% | Glucose (% | Galactose(% | Total carb.* (% | lactose (% | % sugar ** | (AOAC | converted |
| -tration | Sample | w/w) | w/w) | w/w) | w/w) | w/w) | w/v) | reduction | 2001.02 calc) | to TGOS |
| ~4.7% | 60 ° C., | 0.90 | 1.92 | 1.18 | 0.82 | 4.83 | 1.49 | 18.63 | 1.31 | 27.09 |
| lactose | 10 min | | | | | | | | | |
| | 60 ° C., | 0.94 | 1.07 | 1.69 | 1.23 | 4.94 | 0.23 | 18.95 | 1.74 | 35.25 |
| | 40 min | | | | | | | | | |
| | 60 ° C., | 0.95 | 1.02 | 1.94 | 1.43 | 5.34 | 0.14 | 17.73 | 1.82 | 34.16 |
| | 60 min | | | | | | | | | |
| | 60 ° C., | 0.79 | 0.86 | 1.90 | 1.45 | 5.01 | 0.07 | 15.86 | 1.60 | 32.01 |
| | 90 min | | | | | | | | | |
| ~6% | 55° C., | 1.29 | 2.72 | 1.61 | 1.01 | 6.62 | 1.70 | 19.43 | 2.22 | 33.48 |
| lactose | 20 min | | | | | | | | | |
| | 55° C., | 1.39 | 1.82 | 2.06 | 1.33 | 6.59 | 0.56 | 21.04 | 2.59 | 39.24 |
| | 40 min | | | | | | | | | |
| | 55° C., | 1.28 | 1.48 | 2.25 | 1.51 | 6.83 | 0.26 | 18.69 | 2.73 | 39.96 |
| | 60 min | | | | | | | | | |
| | 55° C., | 1.13 | 1.31 | 2.39 | 1.67 | 6.49 | 0.15 | 17.40 | 2.34 | 36.03 |
| | 80 min | | | | | | | | | |
| ~9% | 55° C., | 2.32 | 4.00 | 2.22 | 1.21 | 9.75 | 2.50 | 23.81 | 3.65 | 37.45 |
| lactose | 20 min | | | | | | | | | |
| | 55° C., | 2.54 | 2.85 | 2.86 | 1.62 | 9.87 | 0.82 | 25.77 | 4.40 | 44.58 |
| | 40 min | | | | | | | | | |
| | 55° C., | 2.40 | 2.41 | 3.13 | 1.86 | 9.79 | 0.38 | 24.49 | 4.33 | 44.23 |
| | 60 min | | | | | | | | | |
| | 55° C., | 2.17 | 2.20 | 3.33 | 2.07 | 9.77 | 0.25 | 22.17 | 4.11 | 42.08 |
| | 80 min | | | | | | | | | |
| ~12% | 55° C., | 3.28 | 5.35 | 2.73 | 1.33 | 12.69 | 3.40 | 25.84 | 4.99 | 39.33 |
| lactose | 20 min | | | | | | | | | |
| | 55° C., | 3.59 | 3.81 | 3.46 | 1.76 | 12.62 | 1.20 | 28.44 | 5.95 | 47.13 |
| | 40 min | | | | | | | | | |
| | 55° C., | 3.52 | 3.32 | 3.88 | 2.08 | 12.80 | 0.60 | 27.50 | 6.07 | 47.44 |
| | 60 min | | | | | | | | | |
| | 55° C., | 3.28 | 3.09 | 4.17 | 2.37 | 12.91 | 0.38 | 25.38 | 5.91 | 45.77 |
| | 80 min | | | | | | | | | |
| ~20% | 55° C., | 5.86 | 9.49 | 3.83 | 1.48 | 20.66 | 6.60 | 28.35 | 8.28 | 40.09 |
| lactose | 20 min | | | | | | | | | |
| | 55° C., | 6.84 | 6.95 | 5.07 | 2.10 | 20.96 | 2.80 | 32.61 | 10.44 | 49.80 |
| | 40 min | | | | | | | | | |
| | 55° C., | 6.87 | 6.00 | 5.75 | 2.53 | 21.16 | 1.40 | 32.48 | 11.02 | 52.06 |
| | 60 min | | | | | | | | | |
| | 55° C., | 6.50 | 5.44 | 6.02 | 2.82 | 20.79 | 0.92 | 31.29 | 10.69 | 51.40 |
| | 80 min | | | | | | | | | |
| ~30% | 55° C., | 8.01 | 16.34 | 5.19 | 2.30 | 31.85 | 12.23 | 25.17 | 11.23 | 35.26 |
| lactose | 20 min | | | | | | | | | |
| | 55° C., | 10.37 | 11.60 | 7.07 | 3.11 | 32.14 | 5.52 | 32.26 | 15.26 | 47.49 |
| | 40 min | | | | | | | | | |
| | 55° C., | 10.36 | 9.63 | 7.88 | 3.61 | 31.49 | 4.07 | 32.91 | 15.15 | 48.11 |
| | 60 min | | | | | | | | | |
| | 55° C., | 10.03 | 8.69 | 8.47 | 4.03 | 31.21 | 2.23 | 32.13 | 15.71 | 50.34 |
| | 80 min | | | | | | | | | |

*Total carb. (% w/w) is the sum of DP3+ (% w/w), DP2 (% w/w), glucose (% w/w) and galactose (% w/w)
** % lactose converted to TGOS is calculated as % TGOS relative to total carb.
*** % sugar reduction is % DP3+ relative to Total carb.

Conclusion:

We found that in contrast to the current literature, LBul will not only convert 15% of the lactose (starting at 5.2%) to GOS fibers (DP3+) but more than 18% in a regular milk of 4.7% lactose. This would correspond to more than 18% sugar reduction in a regular Aria Mini-milk. Similarly, we found more than 20% of the lactose converted to DP3+(more than 20% sugar reduction) in a concentrated milk substrate of 6% lactose, more than 25% of the lactose converted to DP3+(more than 25% sugar reduction) in a concentrated milk substrate of 9-12% lactose, more than 30% of the lactose converted to DP3+(more than 30% sugar reduction) in a concentrated milk substrate of 20-40% lactose. Also, in contrast to current literature we found that LBul will not only convert 20% of the lactose (starting at 5.2%) to TGOS but more than 30% in a regular milk of 4.7% lactose. Similarly, we found more than 35% of the lactose converted to TGOS in a concentrated milk substrate of 6% lactose, more than 40% of the lactose converted to TGOS in a concentrated milk substrate of 9% lactose, more than 45% of the lactose converted to TGOS in a concentrated milk substrate of 12% lactose concentrated milk, more than 50% of the lactose converted to TGOS substrate of 20-40% lactose

### Example 20: Lactose Conversion to GOS in 55% Lactose Buffered Substrate

Two solutions of 56.2% (w/w) lactose in either 0.1 M MES pH 6.0 (for LBul) or 50 mM Sodium Acetate pH 4.8 (for NutriBio GOS) was prepared in a volume of 200 ml. The solutions were heated in Heating block with mix, 400 rpm, to >90° C. The solutions were then cooled down at table for about 30 minutes to 59° C. Hereafter, 80 U/ml of NutriBio GOS was added to the lactose solution at pH 4.8 and 0.488 ml of LBul was added to the lactose solution at pH 6 where after the solutions were placed in another heating block at 58° C. The reactions were running for up to 3 hours at 58° C. and 400 rpm. 1 ml of sample was collected from each solution after 0.5, 1, 1.5, 2 and 3 hours and inactivated by heating to 98° C. for 10 minutes. Quantification of DP3+, DP2, glucose, galactose was done according to example 16 and lactose was quantified according to example 2

All data is presented in table 12 together with calculated values of Total carbohydrates (carb.), % sugar reduction, TGOS (according to example 17) and % lactose converted to TGOS.

TABLE 12
LBul and NutriBio GOS lactose conversion to GOS in 55% lactose buffered substrate

| Initial | | | | | | | | | | |
|---|---|---|---|---|---|---|---|---|---|---|
| lactose | | | | | | Total | | | | % lactose |
| concen- | | DP3+ (% | DP2 (% | Glucose (% | Galactose (% | carb. (% | lactose (% | % sugar | TGOS (% w/w) | converted |
| tration | Sample | w/w) | w/w) | w/w) | w/w) | w/w) * | w/v) | reduction ** | (AOAC 2001.02 calc) | to TGOS*** |
| 55% lactose, | 58° C., | 12.01 | 35.27 | 6.43 | 1.96 | 55.66 | 30.00 | 21.57 | 16.17 | 29.05 |
| LBul | 30 min | | | | | | | | | |
| | 58° C., | 16.35 | 26.62 | 9.69 | 3.20 | 55.86 | 19.00 | 29.27 | 22.67 | 40.59 |
| | 60 min | | | | | | | | | |
| | 58° C., | 18.13 | 22.16 | 11.69 | 4.11 | 56.08 | 12.00 | 32.33 | 26.73 | 47.66 |
| | 90 min | | | | | | | | | |
| | 58° C., | 18.19 | 18.64 | 12.75 | 4.78 | 54.36 | 7.90 | 33.46 | 27.54 | 50.66 |
| | 120 min | | | | | | | | | |
| | 58° C., | 18.57 | 16.36 | 15.25 | 6.37 | 56.55 | 4.00 | 32.84 | 29.81 | 52.71 |
| | 180 min | | | | | | | | | |
| 55% lactose, | 58° C., | 14.78 | 31.82 | 7.61 | 1.69 | 55.89 | 32.00 | 26.44 | 15.24 | 27.27 |
| NutriBio | 30 min | | | | | | | | | |
| GOS | 58° C., | 16.50 | 24.67 | 10.73 | 3.03 | 54.93 | 24.00 | 30.04 | 18.55 | 33.77 |
| | 60 min | | | | | | | | | |
| | 58° C., | 16.59 | 23.05 | 11.87 | 3.65 | 55.16 | 21.00 | 30.08 | 20.06 | 33.77 |
| | 90 min | | | | | | | | | |
| | 58° C., | 16.44 | 21.33 | 13.34 | 4.51 | 55.62 | 17.00 | 29.56 | 22.15 | 36.37 |
| | 120 min | | | | | | | | | |
| | 58° C., | 15.38 | 19.10 | 15.13 | 5.82 | 55.44 | 13.00 | 27.74 | 23.12 | 41.70 |
| | 180 min | | | | | | | | | |

* Total carb. (% w/w) is the sum of DP3+ (% w/w), DP2 (% w/w), glucose (% w/w) and galactose (% w/w)
** % lactose converted to TGOS is calculated as % TGOS relative to total carb.
***% sugar reduction is % DP3+ relative to Total carb.

Conclusion

We found that the LBul can produce higher yields of GOS fibers (thereby greater sugar reduction) than the NutriBio GOS used for GOS production today and significantly higher TGOS yields.

### Example 21: Reducing or Eliminating Hold Time for Production of Lactose-Reduced or Lactose-Free High Temperature Short Time (HTST) Milk

Belfonte skim milk was treated with Experimental Dupont lactase enzyme per liter of milk for 6 hours at 4° C. prior to the pasteurization step, or 5 minutes at 4° C. prior to pasteurization step. Milk then went through HTST pasteurization step with 60° C. preheat, 72.8 or 73.3° C. pasteurization for 30 seconds, and 1300 psi homogenization. Milk was cooled and stored at 4° C. for 3 weeks immediately following pasteurization. Sensory evaluation was completed at 3 weeks. Current HTST lactose-reduced or lactose-free conditions typically require 18-24 hours of lactose hydrolysis prior to pasteurization as the enzyme becomes inactivated once it goes through the pasteurizer.

A 1 mL milk sample was taken at multiple timepoints throughout the study and transferred to separate 1.5 mL Eppendorf tubes for lactose quantification according to example 2: initial milk sample before enzymation, at 30 minutes or 2 hours post-pasteurization, at 24-26 hours after pasteurization, at 42-43 hours after pasteurization, and at 8-9 days after pasteurization. When the milk sample was taken, it was immediately placed in a heat block for 10 minutes at 95° C. to inactivate the lactase enzyme. The milk samples were then cooled to room temperature and prepared as follows: 200 μL of the heat-treated milk sample was transferred to a new 1.5 mL Eppendorf tube and 800 μL of deionized water was added and vortexed. 50 μL of Carrez agent I and 50 μL Carrez agent II were added to each tube and vortexed. The solution remained at room temperature for 30 minutes and was then centrifuged for 5 minutes at 3000 rpm. 10 μL of the supernatant was added to 990 μL of deionized water in a separate 1.5 mL Eppendorf tube, vortexed, and filtered through a 0.45 picron filter into a vial for lactose analysis via Ion Chromatography. Lactose concentrations were calculated according to the calibration standards and internal control.

Table 13 shows the enzyme dosage, incubation time prior to pasteurization, and pasteurization temperature for the experiment conducted, along with the milk sample timepoints and lactose results for each sample. The lactose level dropped after pasteurization for all conditions tested and shows the survivability of the enzyme through the pasteurization step and post pasteurization over shelf life. Within 8-9 days post processing, the lactose level achieved <0.1% w/v. No off flavors were detected in any of the samples over the 3-week shelf life. This example illustrates the use of the Experimental DuPont lactase in High Temperature Short Time (HTST) milk to make lactose-free or lactose-reduced milk. The survivability of the enzyme throughout the pasteurization step allows significant reduction or possible elimination of the hold time prior to pasteurization.

TABLE 13

| | | | | Milk Sample | |
|---|---|---|---|---|---|
| | | Hydrolysis | | Timepoint for | |
| | Dosage | Time Prior to | Pasteurization | Lactose | |
| Condition | (g/L) | Pasteurization | Temp (° C.) | Measurement | % W/V Lactose |
| 1 | 1.0 | 6 hours | 72.8 | Initial milk sample | 5.00 |
| | | | | before enzyme | |
| | | | | addition | |
| | | | | 30 minutes post- | 0.33 |
| | | | | pasteurization | |
| | | | | 24 hours post- | 0.21 |
| | | | | pasteurization | |
| | | | | 43 hours post- | 0.16 |
| | | | | pasteurization | |
| | | | | 8 days post- | 0.07 |
| | | | | pasteurization | |
| 2 | 1.25 | 6 hours | 72.8 | Initial milk sample | 5.01 |
| | | | | before enzyme | |
| | | | | addition | |
| | | | | 2 hours post- | 0.10 |
| | | | | pasteurization | |
| | | | | 26 hours post- | 0.08 |
| | | | | pasteurization | |
| | | | | 42 hours post- | 0.07 |
| | | | | pasteurization | |
| | | | | 9 days post- | 0.03 |
| | | | | pasteurization | |
| 3 | 1.0 | 5 minutes | 72.8 | Initial milk sample | 5.00 |
| | | | | before enzyme | |
| | | | | addition | |
| | | | | 30 minutes post- | 2.78 |
| | | | | pasteurization | |
| | | | | 24 hours post- | 0.94 |
| | | | | pasteurization | |
| | | | | 43 hours post- | 0.45 |
| | | | | pasteurization | |
| | | | | 8 days post- | 0.05 |
| | | | | pasteurization | |
| 4 | 1.3 | 5 minutes | 72.8 | Initial milk sample | 5.01 |
| | | | | before enzyme | |
| | | | | addition | |
| | | | | 2 hours post- | 2.53 |
| | | | | pasteurization | |
| | | | | 26 hours post- | 0.56 |
| | | | | pasteurization | |
| | | | | 42 hours post- | 0.21 |
| | | | | pasteurization | |
| | | | | 9 days post- | 0.03 |
| | | | | pasteurization | |
| 5 | 1.6 | 5 minutes | 72.8 | Initial milk sample | 5.01 |
| | | | | before enzyme | |
| | | | | addition | |
| | | | | 2 hours post- | 2.24 |
| | | | | pasteurization | |
| | | | | 26 hours post- | 0.34 |
| | | | | pasteurization | |
| | | | | 42 hours post- | 0.10 |
| | | | | pasteurization | |
| | | | | 9 days post- | 0.02 |
| | | | | pasteurization | |
| 6 | 1.6 | 5 minutes | 73.3 | Initial milk sample | 5.01 |
| | | | | before enzyme | |
| | | | | addition | |
| | | | | 2 hours post- | 2.55 |
| | | | | pasteurization | |
| | | | | 26 hours post- | 1.00 |
| | | | | pasteurization | |
| | | | | 42 hours post- | 0.52 |
| | | | | pasteurization | |
| | | | | 9 days post- | 0.01 |
| | | | | pasteurization | |

### REFERENCES

1: EP303201
2: Fischer, C. and Kleinschmiidt, T. (2018), Synthesis of Galactooligosaccharides in Milk and Whey: A Review. Comprehensive Reviews in Food Science and Food Safety, 17: 678-697.
3: Tien-Thanh Nguyen, Hoang Anh Nguyen, Sheryl Lozel Arreola, Georg Mlynek, Kristina Djinović-Carugo, Geir Mathiesen, Thu-Ha Nguyen, and Dietmiar Haltrich. Homodimeric β-Galactosidase from Lactobacillus delbrueckii subsp. bulgaricus DSM 20081: Expression in Lactobacillus plantarumi and Biochemical Characterization. Journal of Agricultural and Food Chemistry 2012 60 (7), 1713-1721. DOI: 10.1021/jf203909e
4: Todor Vasiljevic and Paul Jelen (2003). Oligosaccharide production and proteolysis during lactose hydrolysis using crude cellular extracts from lactic acid bacteria. Lait, 83 6 (2003) 453-467.

All references cited in this specification are incorporated by reference into this specification.

1. A method for reducing the amount of lactose in a milk-based substrate containing lactose, wherein said method comprises contacting said substrate with an enzyme having neutral lactase activity at a temperature of more than about 50° C., and wherein said lactase reduces the amount of lactose in said substrate by at least about 70%.

2. The method according to claim 1 wherein said enzyme having neutral lactase activity is derived from a Lactobacillus.

3. The method according to claim 1 or claim 2 wherein said enzyme having neutral lactase activity is derived from Lactobacillus delbrueckii bulgaricus.

4. The method according to any one of claims 1 to 3 wherein said enzyme has at least about 60% identity to SEQ ID NO:1.

5. The method according to any one of claims 1 to 4 wherein said enzyme has at least about 60, 65, 70, 75, 80, 85, 90, 91, 92, 93, 94, 95, 96, 97, 98, 99 or 100% identity to SEQ ID NO:1.

6. The method according to any one of claims 1 to 5, wherein said enzyme is that of SEQ ID NO:1, or a lactase active fragment thereof.

7. The method according to any one of claims 1 to 6 wherein said enzyme having neutral lactase activity is purified.

8. The method according to any one of claims 1 to 7 wherein said enzyme having neutral lactase activity is concentrated.

9. The method according to any one or claims 1 to 8 wherein said enzyme having neutral lactase activity is in the form of an enzyme preparation which has a reduced level of lipase side activity.

10. The method according to claim 9 wherein said enzyme having neutral lactase activity is in the form of an enzyme preparation which has a reduced level of protease, amylase, mannanase, pectinase, cellulase and/or p-nitrobenzylesterase side activities.

11. The method according to claim 9 or claim 10 wherein said preparation is substantially free of lipase, protease, amylase, mannanase, pectinase, cellulase and/or p-nitrobenzylesterase side activities.

12. The method according to any one of claims 1 to 11 wherein said temperature is about 50° C. to about 65° C., preferably about 55° C. to about 60° C.

13. The method according to any one of claims 1 to 12 wherein said temperature is selected from about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 and 90° C.

14. The method according to any one of claims 1 to 13 wherein said temperature is about 55 or 58° C.

15. The method according to any one of claims 1 to 14 wherein the activity of the enzyme at 60° C. is at least 50% of the activity at 50° C.

16. The method according to any of one of claims 1 to 15 wherein said enzyme has an optimum temperature of about 60° C.

17. The method according to any of one of claims 1 to 16 wherein said enzyme has an optimum pH of about 5.5 to about 8.0.

18. The method according to any of one of claims 1 to 17, wherein the lactase activity of said enzyme at pH 6.0 is at least 50% of its lactase activity at pH 6.5 when measured at 37° C.

19. The method according to any one of claims 1 to 18 wherein said contacting is performed between about 10 minutes and about 4 hours.

20. The method according to any one of claims 1 to 19 wherein said milk-based substrate is selected from solutions/suspensions of any milk or milk like products comprising lactose, such as whole or low fat milk, skim milk, buttermilk, reconstituted milk powder, condensed milk, solutions of dried milk, UHT milk, whey, whey permeate, acid whey, or cream.

21. The method according to any one of claims 1 to 20 wherein said milk-based substrate is raw milk which is not pasteurized before contact with said enzyme.

22. The method according to any one of claims 1 to 21 where said substrate comprises about 3% to 30% lactose, preferably about 3% to 16% lactose, more preferably about 4% to 5% lactose.

23. The method according to any one of claims 1 to 22 wherein said amount of lactose is reduced by at least about 70, 75, 80, 85, 90, 91, 92, 93, 94, 95, 96, 97, 98, 99 or 100%, preferably at least about 97%.

24. The method according to any one of claims 1 to 23 wherein said lactose is reduced to below 100 ppm or below 1000 ppm.

25. The method according to claim 24 wherein said lactose is reduced to below 100 ppm at a temperature over 50° C. within 180 minutes, preferably 120 minutes.

26. The method according to any one of claims 1 to 25 wherein said enzyme is added to the milk-based substrate at a concentration of about 24 to about 240 NLU per g lactose.

27. The method according to any one of claims 1 to 26 wherein said enzyme has a ratio of lactase:transgalatosylase activity of more than 1:1, or wherein said enzyme has a ratio of transgalactosylating activity below 120%.

28. The method according to any one of claims 1 to 27 wherein said enzyme has a ratio of lactase activity above 100%.

29. A milk-based substrate with reduced lactose content obtained or obtainable by the method according to any one of claims 1 to 28.

30. A method according to any one of claims 1 to 28 for producing a dairy product.

31. The method according to claim 30 wherein said dairy product is selected from skim milk, low fat milk, whole milk, cream, UHT milk, milk having an extended shelf life, a fermented milk product, cheese, yoghurt, butter, dairy spread, butter milk, acidified milk drink, sour cream, whey based drink, condensed milk, dulce de leche, a flavoured milk drink, sweetened condensed milk, milk powder, reconstituted dairy products, ice-cream, Ryazhenka, pudding, desserts and milk-shakes.

32. The method according to claim 31 wherein said dairy product is condensed milk, ice cream or milk-shake.

33. The method according to any one of claims 1 to 28 and 30 to 32 wherein said method includes a pasteurization step.

34. A dairy product obtained or obtainable by the method according to any one of claims 30 to 33.

35. The method according to any one of claims 30 to 33 wherein said dairy product is condensed milk and said temperature is about 55° C.

36. An enzyme preparation comprising an enzyme having neutral lactase activity, wherein said enzyme can reduce the amount of lactose in a substrate by at least 70% at a temperature of 50° C. or more, 37. The enzyme preparation according to claim 36 wherein said enzyme is not that of SEQ ID NO:1.

38. The enzyme preparation according to claim 36 or 37 wherein said enzyme having neutral lactase activity is purified, wherein preferably the enzyme is that of SEQ ID NO:1 or a lactase active fragment thereof.

39. The enzyme preparation according any one of claims 36 to 38 wherein said enzyme having neutral lactase activity is concentrated.

40. The enzyme preparation according any one of claims 36 to 39 wherein said enzyme having neutral lactase activity has at least about 60, 65, 70, 75, 80, 85, 90, 91, 92, 93, 94, 95, 96, 97, 98 or 99% identity to SEQ ID NO:1.

41. The enzyme preparation according to any one of claims 36 to 40, wherein said enzyme preparation has a reduced level of lipase side activity.

42. The enzyme preparation according to any one of claims 36 to 41, wherein said enzyme preparation has a reduced level of protease, amylase, mannanase, pectinase, cellulase and/or p-nitrobenzylesterase side activities.

43. The enzyme preparation according to claim 41 or 42, wherein said enzyme preparation is substantially free of lipase, protease, amylase, mannanase, pectinase, cellulase and/or p-nitrobenzylesterase side activities.

44. The enzyme preparation according to any one of claims 36 to 43 wherein the activity of the enzyme having neutral lactase activity at 60° C. is at least 50% of the activity at 50° C.

45. The enzyme preparation according to any one of claims 36 to 44 wherein said enzyme having neutral lactase activity has an optimum temperature of about 60° C.

46. The enzyme preparation according to any one of claims 36 to 45 wherein said enzyme having neutral lactase activity has an optimum pH of about 5.5 to about 8.0.

47. The enzyme preparation according to any one of claims 36 to 46 wherein said enzyme having neutral lactase activity at pH 6.0 is at least 50% of its lactase activity at pH 6.5 when measured at 37° C.

48. A nucleic acid molecule encoding an enzyme having neutral lactase activity or lactase active fragment thereof as defined in any one of claims 36 to 47.

49. An expression vector comprising a nucleic acid molecule according to claim 48, or capable of expressing an having neutral lactase activity or lactase active fragment thereof as defined in any one of claims 36 to 47.

50. A cell capable of expressing an enzyme having neutral lactase activity or lactase active fragment thereof as defined in any one of claims 36 to 47.

51. A method of expressing an enzyme, comprising providing a cell according to claim 50 and expressing the enzyme from the cell, and optionally purifying the enzyme.

52. An enzyme having neutral lactase activity as defined in any one of claims 36 to 47 which has a half-life in milk of more than 4 hours at 55° C., or more than 1 hour at 58° C.

53. Use of an enzyme preparation according to any one of claims 36 to 47 for preparing a dairy product.

54. The use according to claim 53 wherein said dairy product is selected from skim milk, low fat milk, whole milk, cream, UHT milk, milk having an extended shelf life, a fermented milk product, cheese, yoghurt, butter, dairy spread, butter milk, acidified milk drink, sour cream, whey based drink, condensed milk, dulce de leche, a flavoured milk drink, sweetened condensed milk, milk powder, reconstituted dairy products, ice-cream, ice-cream, Ryazhenka, pudding, dessert and milk-shakes.

55. A bacterial expression host capable of expressing an enzyme having neutral lactase activity or a lactase active fragment thereof as defined in any one of claims 36 to 47 wherein the host cell comprises a genetic modification which reduces or eliminates lipase activity, preferably lipase A activity.

56. The bacterial expression host according to claim 55 wherein the genetic modification comprises a deletion, disruption or down-regulation of a gene encoding a Lipase A polypeptide as set out in SEQ ID NO:2.

57. A bacterial expression host capable of expressing an enzyme having neutral lactase activity as defined in any one of claims 36 to 47 wherein the host cell comprises a genetic modification which reduces or eliminates one or more enzyme activity selected from: protease, amylase, mannanase, pectinase, cellulase and p-nitrobenzylestaerase activities.

58. The bacterial expression host according to claim 56 or claim 57 wherein the host cell comprises a genetic modification which reduces or eliminates lipase, protease, amylase, mannanase, pectinase, cellulase and p-nitrobenzylestaerase activities.

59. The bacterial expression host according to any one of claims 55 to 58 wherein said bacterium is a Bacillus sp.

60. The bacterial expression host according to claim 59, selected from the group consisting of B. subtilis, B. lichenmformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii, and B. thuringiensis.

61. The bacterial expression host according to claim 60 wherein said bacterium is a Bacillus subtilis.

62. The bacterial expression host according to any one of claims 55 to 61 wherein said bacterium comprises a nucleic acid molecule according to claim 48 or an expression vector according to claim 49.

63. The bacterial expression host according to any one of claims 55 to 62, wherein said host expresses an enzyme having neutral lactase activity.

64. An enzyme having neutral lactase activity produced by the bacterial expression host according to claim 63.

65. An enzyme according to claim 64 wherein said enzyme is as defined in any one of claims 37 to 47.

66. A dairy product comprising the bacterial expression host according to any one of claims 55 to 65.

67. Use of the bacterial expression host according to any one of claims 55 to 65 for preparing a dairy product.

68. A method for producing a lactose reduced or lactose free milk shake, ice cream, reconstituted milk product, desserts, pudding, condensed milk, sweetened condensed milk, Ryazhenka, Dulce de Leche or milk based powder, said method comprising contacting a milk-based substrate with an enzyme having neutral lactase activity lactase at 50-65° C.

69. A method for producing milk-based powder or whey powder with a reduced lactose content in which an enzyme having neutral lactase activity is added for hydrolyzation during the evaporation or condensing process.

70. A method for production of a lactose free dairy product from a milk-based substrate with an enzyme having neutral lactase activity, wherein more than 20% activity remains in the milk-based substrate after pasteurization at 72° C. for 15 seconds.

71. A method for producing a fermented dairy product comprising adding an enzyme having neutral lactase activity after pasteurization and homogenization at 50-65° C.

72. The method of claim 71 further comprising cooling to about 45° C. and adding starter cultures.

73. A method for in situ GOS production in a milk-based substrate comprising at least 4.7% (w/w) lactose, comprising contacting said substrate with an enzyme having neutral lactase activity wherein more than 30% of said lactose is converted into TGOS

74. The method of claim 73 wherein the milk-based substrate comprises between 6-40% lactose (w/w) and wherein more than 40% of the lactose is converted into TGOS.

75. A method for reducing the amount of sugar in a milk-based substrate comprising at least 4.7% (w/w) lactose, comprising contacting said substrate with an enzyme having neutral lactase activity wherein more than 18% of said lactose is converted into GOS fibers (DP3+).

76. The method of claim 75 wherein the milk-based substrate comprises at least 6-9% lactose and wherein more than 20% of the lactose is converted into GOS fibers (DP3+).

77. The method of claim 76 wherein the milk-based substrate comprises at least 9-20% lactose and wherein more than 25% of the lactose is converted into GOS fibers (DP3+).

78. The method of claim 77 wherein the milk-based substrate comprises at least 20-40% lactose and wherein more than 30% of the lactose is converted into GOS fibers (DP3+).

79. The method of claim 78 wherein the milk-based substrate comprises at least 40-65% lactose and wherein more than 30% of the lactose is converted into GOS fibers (DP3+).

80. The method of claim 79 wherein the milk-based substrate comprises at least 40-65% lactose and wherein more than 40% of the lactose is converted into GOS fibers (DP3+).

81. A method for production of a lactose free dairy product from a milk-based substrate comprising:
a.) providing a milk-based substrate;
b.) adding an enzyme having neutral lactase activity to the milk-based substrate;
c.) pasteurizing the milk-based substrate wherein the lactase having neutral lactase activity retains a substantial amount of activity after said pasteurizing step; and
d.) storing the milk-base substrate for a sufficient time to provide a lactose free dairy product.

82. The method of claim 81 wherein the milk-based substrate comprises at least 4.7% (w/w) lactose.

83. The method of any of claim 81 or 82 wherein the pasteurizing step is carried out at 65 to 75° C.

84. The method of claim 83 wherein the pasteurizing step is carried out at 70 to 73° C.

85. The method of claim 84 wherein the pasteurizing step is carried out at 72.8° C.

86. The method of any of claims 81 to 85 wherein the step of storing is for 3 to 10 days.

87. The method of any of claim 86 wherein the step of storing is 6 to 9 days.

88. The method of claim 87 wherein the step of storing is 8 days.

89. The method of any of claims 68 to 88 wherein said enzyme having neutral lactase activity is derived from a Lactobacillus.

90. The method of claim 89 wherein said enzyme having neutral lactase activity is derived from Lactobacillus delbrueckii bulgaricus.

91. The method according to 90 wherein said enzyme has at least about 60% identity to SEQ ID NO:1.

92. The method of claim 91 wherein said enzyme has at least about 60, 65, 70, 75, 80, 85, 90, 91, 92, 93, 94, 95, 96, 97, 98, 99 or 100% identity to SEQ ID NO:1.

93. The method of claim 92 wherein said enzyme is that of SEQ ID NO:1 or a lactase active fragment thereof.

94. The method of claim 93 wherein said enzyme having neutral lactase activity is purified.

95. The method of claim 94 wherein said enzyme having neutral lactase activity is concentrated.

96. The method of claim 95 wherein said enzyme having neutral lactase activity is in the form of an enzyme preparation which has a reduced level of lipase side activity.

97. The method of claim 96 wherein said enzyme having neutral lactase activity is in the form of an enzyme preparation which has a reduced level of protease, amylase, mannanase, pectinase, cellulase and/or p-nitrobenzylesterase side activities.

98. The method of claim 97 wherein said preparation is substantially free of lipase, protease, amylase, mannanase, pectinase, cellulase and/or p-nitrobenzylesterase side activities.
